(19) United States
(12) Patent Application Publication
(10) Pub. No.: US 2026/0269005 A1
(43) Pub. Date: Sep. 10, 2026

(54) UTILIZING MASKED AUTOENCODER GENERATIVE MODELS TO EXTRACT CELLULAR RESPONSE REPRESENTATION EMBEDDINGS

(71) Applicant: Recursion Pharmaceuticals, Inc., Salt Lake City, UT (US)

(72) Inventors: Ihab BENDIDI, Paris (FR); Imran Saeedul HAQUE, Salt Lake City, UT (US); Jason Siyanda HARTFORD, London (GB); John Samuel Hong URBANIK, Brooklyn, NY (US); Juan Sebastián RODRÍGUEZ VERA, Riverton, UT (US); Kian Runnels KENYON-DEAN, Toronto (CA); Konstantin DONHAUSER, Oberland (LI); Marta Marie FAY, Salt Lake City, UT (US); Mohammadsadegh SABERIAN, Kitchener (CA); Nil SAHIN, Toronto (CA); Oren Zeev KRAUS, Toronto (CA); Safiye CELIK, Nashua, NH (US); Zitong WANG, Mississauga (CA)

(21) Appl. No.: 19/074,095

(22) Filed: Mar. 7, 2025

(51) Int. Cl. G16B 5/00; G06F 30/27; G16B 40/20

(52) U.S. Cl. CPC G16B 5/00; G06F 30/27; G16B 40/20

(57) ABSTRACT

The present disclosure relates to systems, non-transitory computer-readable media, and methods for training and utilizing generative machine learning models to generate embeddings from cellular response representations. For example, the disclosed systems can train a generative machine learning model (e.g., a masked autoencoder generative model) to generate predicted (or reconstructed) phenomic images from masked version of ground truth training phenomic images. Moreover, the disclosed systems can filter training cellular response representation data utilizing perturbation significances identified from machine learning embeddings of the training cellular response representation data to generate a focused set of training cellular response representations. Additionally, the disclosed systems can further fine-tune a subset of parameters of the generative machine learning model (trained for the cellular response representation completion task) utilizing a perturbation classification task. In addition, the disclosed systems can further utilize linear probing models to generate improved cellular response representation embeddings from a selected intermediate layer.

### BACKGROUND

Recent years have seen significant improvements in hardware and software platforms for utilizing computing devices to extract and analyze digital signals corresponding to biological relationships. For example, existing systems often utilize computer-based models to extract latent features from images portraying cells. In addition, conventional systems often conduct analyses of the features extracted from cell images to determine biological (or chemical) relationships from the images. Indeed, existing systems often infer biological relationships from cellular phenotypes in high-content microscopy screens by using deep vision models to capture biological signals. Although conventional systems can utilize computer-based models to extract and analyze digital signals for images portraying cells, these conventional systems often have a number of technical deficiencies with regard to computational inefficiencies, extraction inaccuracies, and inflexibilities in training and utilizing machine learning to extract features (or digital signals) from microscopy images.

### SUMMARY

Embodiments of the present disclosure provide benefits and/or solve one or more of the foregoing or other problems in the art with systems, non-transitory computer-readable media, and computer-implemented methods for training and utilizing generative machine learning models to generate embeddings from cellular response representations (e.g., phenomic images, transcriptomic profiles). For instance, the disclosed systems can train a generative machine learning model to generate predicted cellular response representations from masked versions of ground truth training cellular response representations (e.g., a cellular response representation completion task). In addition, the disclosed systems can utilize the trained generative machine learning model to generate cellular response representation embeddings from input cellular response representations (for various phenomic comparisons and/or other data analyses in an experiment design).

In one or more embodiments, the disclosed systems filter training cellular response representation data utilizing perturbation significances identified from machine learning embeddings of the training cellular response representation data to generate a focused set of training cellular response representations to improve the training accuracy of the generative machine learning model. Moreover, the disclosed systems can further improve the performance of the generative machine learning model by fine-tuning a subset of parameters of the generative machine learning model (trained for the cellular response representation completion task) utilizing a perturbation classification task. In addition, the disclosed systems can further utilize linear probing models to identify intermediate layers from the generative machine learning model to generate improved cellular response representation embeddings from a selected intermediate layer(s).

Additional features and advantages of one or more embodiments of the present disclosure are outlined in the description which follows, and in part can be determined from the description, or may be learned by the practice of such example embodiments.

### BRIEF DESCRIPTION OF THE DRAWINGS

The detailed description is described with reference to the accompanying drawings in which:

FIG. 1 illustrates an overview of a perturbation autoencoder modeling system training a generative machine learning model utilizing filtered cellular response representation data in accordance with one or more embodiments.

FIG. 2 illustrates an overview of a perturbation autoencoder modeling system utilizing linear probing to select an inference execution layer to generate perturbation embeddings in accordance with one or more embodiments.

FIG. 3 illustrates an exemplary flow of a perturbation autoencoder modeling system training a generative machine learning model to generate cellular response representation embeddings from cellular response representations in accordance with one or more embodiments.

FIG. 4 illustrates a perturbation autoencoder modeling system filtering training cellular response representation data utilizing perturbation significance metrics identified from machine learning embeddings in accordance with one or more embodiments.

FIGS. 5A and 5B illustrate a perturbation autoencoder modeling system training a machine learning model utilizing both a cellular response representation completion task and a perturbation classification task in accordance with one or more embodiments.

FIGS. 6A and 6B illustrate a perturbation autoencoder modeling system utilizing linear probing to select an inference execution layer from intermediate layers of a generative machine learning model in accordance with one or more embodiments.

FIG. 7 illustrates a perturbation autoencoder modeling system utilizing generated perturbation embeddings in accordance with one or more embodiments.

FIG. 8 illustrates an exemplary graphical user interface for displaying a perturbation similarity heatmap of perturbation comparisons in accordance with one or more embodiments.

FIG. 9 illustrates an exemplary flow of a perturbation autoencoder modeling system generating a corrected phenomic image using a generative machine learning model in accordance with one or more embodiments.

FIGS. 10A, 10B, 11, 12, 13, and 14 illustrate experimental results of utilizing a generative machine learning model in accordance with one or more embodiments.

FIG. 15 illustrates a schematic diagram of a system environment in which a perturbation autoencoder modeling system can operate in accordance with one or more embodiments.

FIG. 16 illustrates an example series of acts for training a generative machine learning model to generate cellular response representation embeddings in accordance with one or more embodiments.

FIG. 17 illustrates an example series of acts for selecting an inference execution layer to generate cellular response representation embeddings from a masked autoencoder generative model in accordance with one or more embodiments.

FIG. 18 illustrates a block diagram of an example computing device for implementing one or more embodiments of the present disclosure.

### DETAILED DESCRIPTION

This disclosure describes one or more embodiments of a perturbation autoencoder modeling system that generates embeddings from phenomic microscopy images (or other cellular response representations) using a generative machine learning model. In one or more implementations, the perturbation autoencoder modeling system trains a generative machine learning model (e.g., a masked autoencoder generative model) to generate reconstructed phenomic images from training masked phenomic images. Indeed, the perturbation autoencoder modeling system further utilizes the trained generative machine learning model to generate perturbation embeddings (e.g., embeddings of cellular response representation portraying perturbations) for input phenomic microscopy images.

To improve the performance of generating accurate perturbation embeddings from the generative machine learning model, in one or more embodiments, the perturbation autoencoder modeling system utilizes training cellular response representation data filtering based on perturbation significance metrics identified from machine learning embeddings of the training cellular response representation data to generate a focused set of training cellular response representations for the generative model. In addition, in one or more embodiments, the perturbation autoencoder modeling system also trains a subset of parameters of the generative machine learning model utilizing a perturbation classification task (e.g., after training the generative machine learning model via the cellular response representation completion task). Moreover, in one or more embodiments, the perturbation autoencoder modeling system also utilizes linear probing to select an inference execution layer(s) from intermediate layers of the generative machine learning model to generate perturbation embeddings with improved accuracy via the inference execution layer.

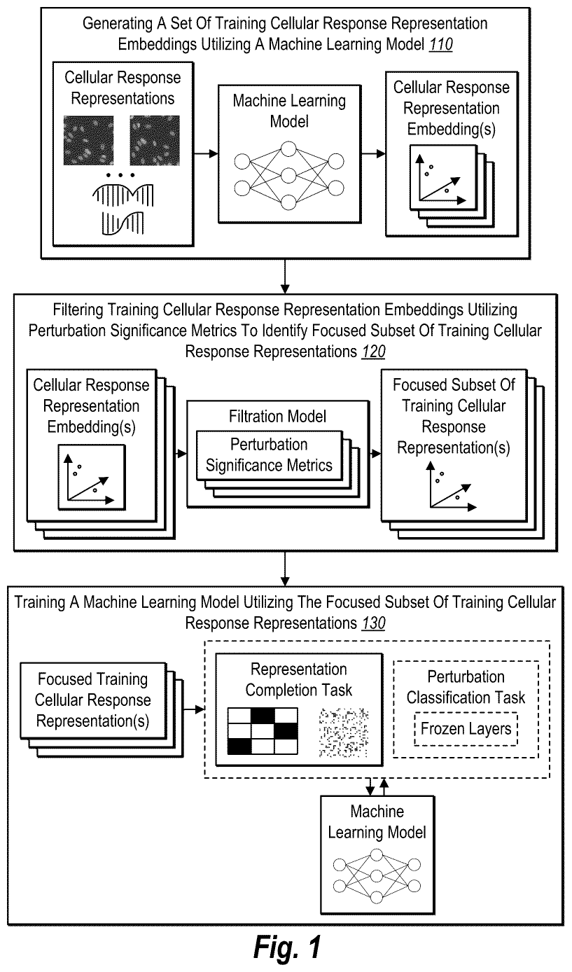

For example, FIG. 1 illustrates an overview of a perturbation autoencoder modeling system 106 training a generative machine learning model utilizing cellular response representation data. Indeed, FIG. 1 illustrates an overview of the perturbation autoencoder modeling system 106 training a generative machine learning model (e.g., a masked autoencoder generative model) to generate reconstructed cellular response representations from training masked cellular response representations to utilize the generative machine learning model to generate cellular response representation embeddings from input cellular response representations. In particular, FIG. 1 illustrates the perturbation autoencoder modeling system 106 filtering training cellular response representation data utilizing perturbation significance metrics identified from machine learning embeddings of the training cellular response representation data to generate a focused set of training cellular response representations for training of the generative machine learning model. In addition, FIG. 1 also illustrates the perturbation autoencoder modeling system 106 fine-tuning a subset of parameters of the generative machine learning model (trained for the cellular response representation completion task) utilizing a perturbation classification task.

In particular, as shown in FIG. 1, the perturbation autoencoder modeling system 106 generates a set of training cellular response representation embeddings utilizing a machine learning model, filters the training cellular response representation embeddings utilizing perturbation significance values to identify a focused subset of training cellular response representation embeddings, and trains a machine learning model utilizing the focused subset of training cellular response representation embeddings. Indeed, the perturbation autoencoder modeling system 106 can accurately remove (or weight) unhelpful training cellular response representation samples from a set of training cellular response representations. As an example, the perturbation autoencoder modeling system 106 can remove (or filter) training cellular response representations where a perturbation fails to result in a noticeable expression (e.g., a visual expression or a gene count expression). Indeed, the perturbation autoencoder modeling system 106 can filter training cellular response representations that encode similar information to a control sample of cellular response representations (via training cellular response representation embeddings generated from the set of training cellular response representations).

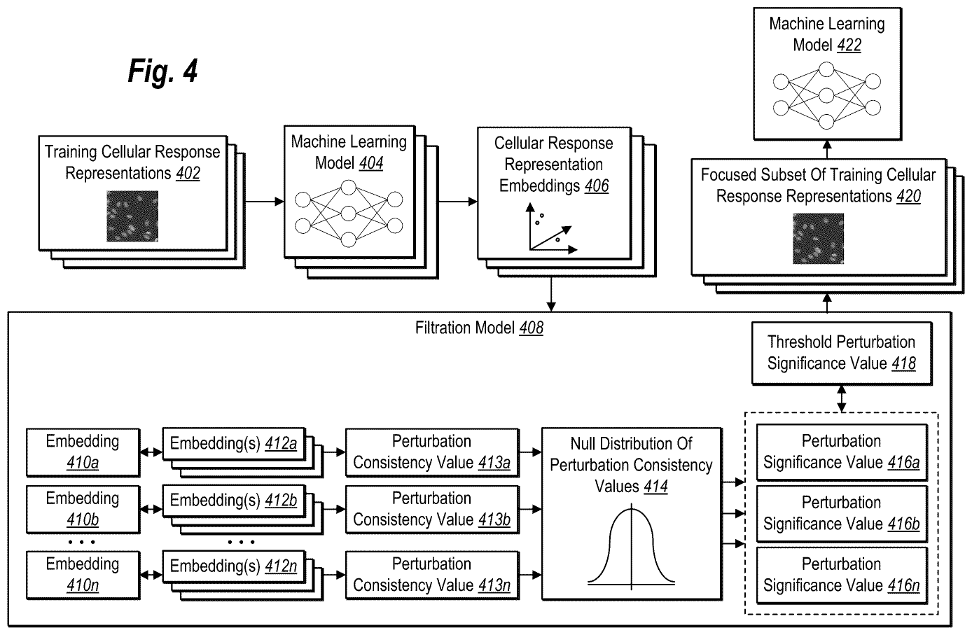

For example, as shown in an act 110 of FIG. 1, the perturbation autoencoder modeling system 106 generates a set of training cellular response representation embeddings utilizing a machine learning model. Indeed, in one or more implementations, the perturbation autoencoder modeling system 106 generates (or identifies) training cellular response representation embeddings by utilizing a machine learning model to embed training cellular response representations in a low-dimension feature space (e.g., as vector representations). For example, the perturbation autoencoder modeling system 106 can utilize embeddings generated within one or more layers of a masked autoencoder generative model and/or a perturbation classification neural network to generate training cellular response representation embeddings from input training cellular response representations. For instance, the perturbation autoencoder modeling system 106 can generate training cellular response representation embeddings as described in greater detail below (e.g., in reference to FIG. 4).

In addition, as shown in an act 120 of FIG. 1, the perturbation autoencoder modeling system 106 filters training microscopy embeddings utilizing perturbation significance metrics to identify a focused subset of training cellular response representation(s). For example, the perturbation autoencoder modeling system 106 can generate perturbation significance metrics for the cellular response representations relative to sampled subsets of the cellular response representations. Indeed, the perturbation autoencoder modeling system 106 can generate a perturbation consistency value for a perturbation by determining a combined similarity measure between a cellular response representation (of the perturbation) and a subset of cellular response representations corresponding to the same or similar perturbation). Moreover, the perturbation autoencoder modeling system 106 can generate the perturbation significance metric by comparing the perturbation consistency value to a null distribution of perturbation consistency values of randomly selected training cellular response representations. Indeed, the perturbation autoencoder modeling system 106 can utilize the generated perturbation significance metric to filter a set of training cellular response representation embeddings (e.g., via a comparison to a threshold perturbation significance metric) to generate a focused subset of training cellular response representations (e.g., based on the corresponding embeddings). Indeed, the perturbation autoencoder modeling system 106 can filter training microscopy embeddings utilizing perturbation significance metrics as described in greater detail below (e.g., in reference to FIG. 4).

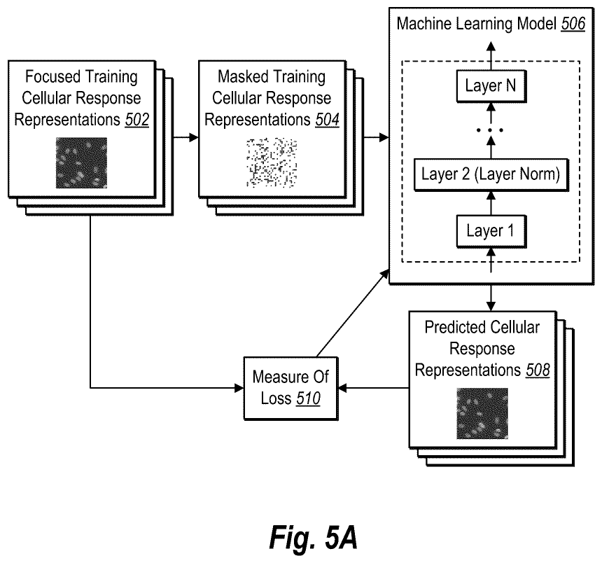

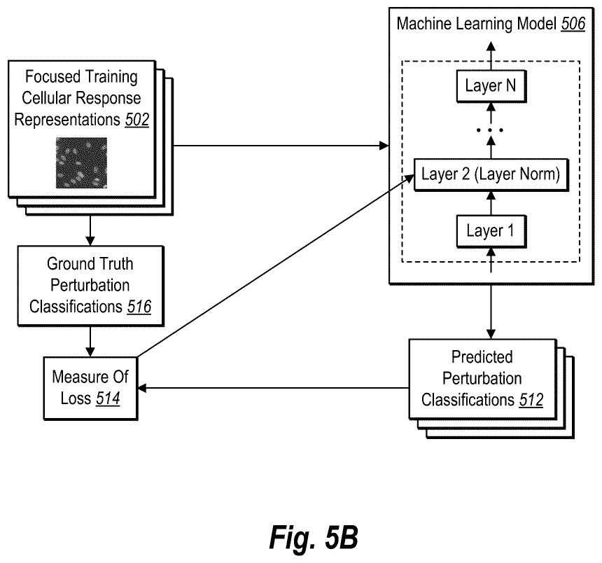

Moreover, as shown in an act 130 of FIG. 1, the perturbation autoencoder modeling system 106 trains a machine learning model utilizing the focused subset of training cellular response representation(s). As shown in FIG. 1, the perturbation autoencoder modeling system 106 can train a machine learning model (e.g., a generative machine learning model) for a cellular response representation completion task using masked versions of the focused training cellular response representations corresponding to the filtered subset of training cellular response representation embeddings. Indeed, the perturbation autoencoder modeling system 106 can train the machine learning model utilizing a cellular response representation completion task on focused training cellular response representations as described in greater detail below (e.g., in reference to FIGS. 5A and 5B).

Furthermore, in some embodiments, as shown in FIG. 1, the perturbation autoencoder modeling system 106 can further fine-tune (or train) a subset of parameters of the machine learning model (trained for the cellular response representation completion task) utilizing a perturbation classification task. For example, the perturbation autoencoder modeling system 106 can initially train the generative machine learning model on the cellular response representation completion task and freeze the pre-trained layers of the generative machine learning model. Moreover, the perturbation autoencoder modeling system 106 can unfreeze a subset of the layers (e.g., parameters of one or more layer norms) to modify the parameters of the subset of the layers on a perturbation classification task. Indeed, the perturbation autoencoder modeling system 106 can train the machine learning model utilizing a cellular response representation completion task (and, subsequently, utilizing a perturbation classification task on a subset of layers) as described in greater detail below (e.g., in reference to FIGS. 5A and 5B).

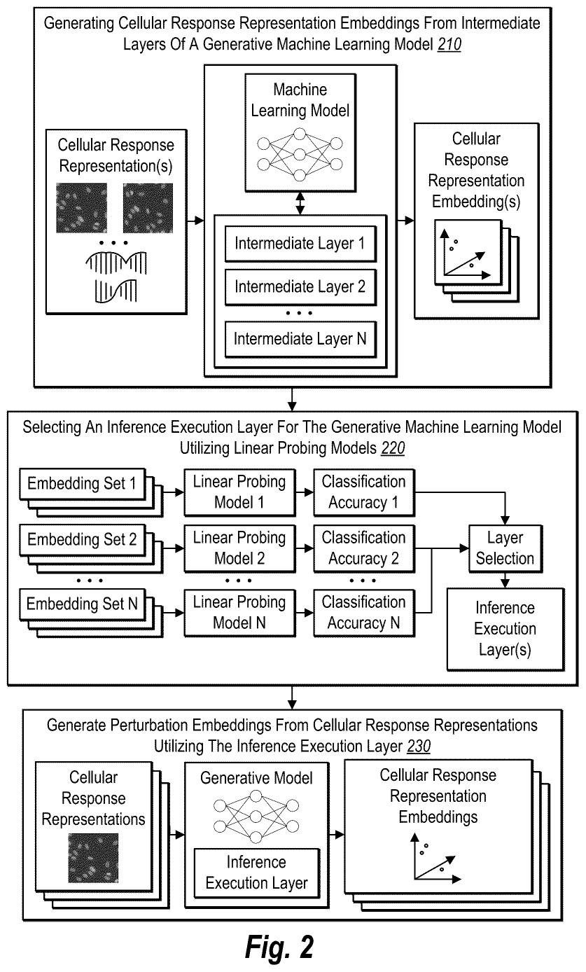

Moreover, FIG. 2 illustrates an overview of the perturbation autoencoder modeling system 106 utilizing linear probing to select an inference execution layer from intermediate layers of the generative machine learning model to generate perturbation embeddings with improved accuracy via the inference execution layer. For instance, as shown in FIG. 2, the perturbation autoencoder modeling system 106 generates cellular response representation embeddings from intermediate layers of a generative machine learning model, selects an inference execution layer for the generative machine learning model utilizing linear probing models, and generates perturbation embeddings from input cellular response representations utilizing the inference execution layer of the generative machine learning model.

Indeed, the perturbation autoencoder modeling system 106 can utilize linear probing to search for intermediate layers of the generative machine learning model to generate improved perturbation embeddings. Specifically, the perturbation autoencoder modeling system 106 can build a set of linear probing models (e.g., logistic regression models) on output features of each transformer block of the generative machine learning model to predict a perturbation classification from the output features. In addition, the perturbation autoencoder modeling system 106 can select the intermediate block (or layer) based on perturbation classification accuracy metrics of the output features (e.g., perturbation embeddings) from the perturbation classifications (e.g., a test balance accuracy for generating the perturbation embeddings).

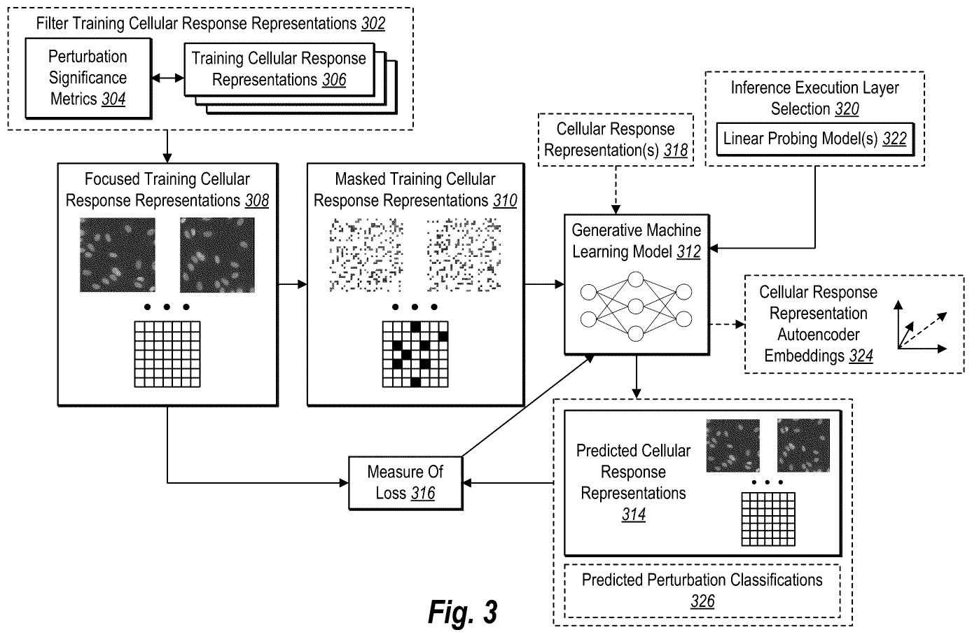

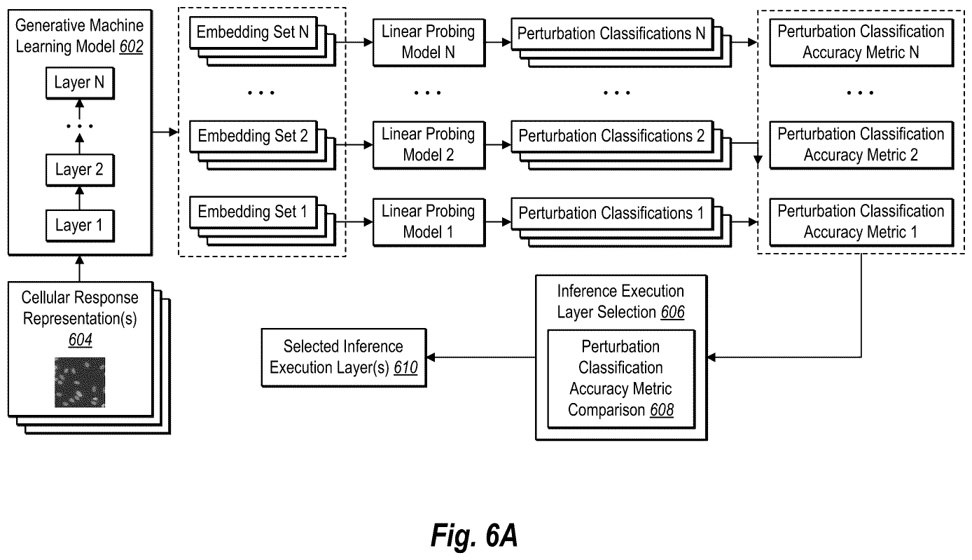

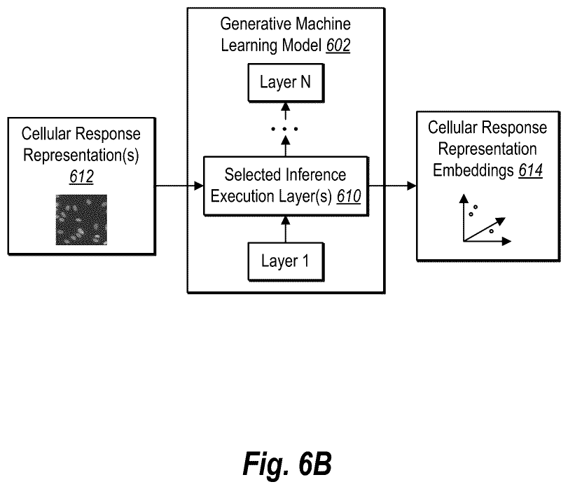

To illustrate, as shown in an act 210 of FIG. 2, the perturbation autoencoder modeling system 106 generates cellular response representation embeddings from intermediate layers of a generative machine learning model. In particular, the perturbation autoencoder modeling system 106 utilizes input cellular response representation(s) with a machine learning model to generate (or extract) cellular response representation embeddings from intermediate layers of the machine learning model. Indeed, the perturbation autoencoder modeling system 106 can generate cellular response representation embeddings from intermediate layers of a machine learning model as described in greater detail below (e.g., in reference to FIGS. 3, 6A, and 6B).

Furthermore, as shown in an act 220 of FIG. 2, the perturbation autoencoder modeling system 106 selects an inference execution layer for the generative machine learning model utilizing linear probing models. In particular, the perturbation autoencoder modeling system 106 can utilize linear probing models trained to generate perturbation classifications from cellular response representation embeddings to generate perturbation classifications from different sets of cellular response representation embeddings extracted from intermediate layers of a machine learning model (e.g., embedding sets). Furthermore, the perturbation autoencoder modeling system 106 can utilize the perturbation classifications corresponding to each set of cellular response representation embeddings to determine a classification accuracy of the particular set of cellular response representation embeddings from the perturbation classifications (e.g., a test balance accuracy for generating the perturbation embeddings) at each intermediate layer. Moreover, the perturbation autoencoder modeling system 106 can utilize the determined classification accuracies to identify (or select) a layer of the machine learning model (as the inference execution layer(s)) that generates cellular response representation embeddings that achieve a particular classification accuracy (e.g., a highest test balance accuracy). Indeed, the perturbation autoencoder modeling system 106 can select an inference execution layer(s) for the generative machine learning model utilizing linear probing models as described in greater detail below (e.g., in reference to FIGS. 6A and 6B).

Moreover, as shown in an act 230 of FIG. 2, the perturbation autoencoder modeling system 106 generates perturbation embeddings from input cellular response representations utilizing the inference execution layer of the generative machine learning model. In particular, upon selecting (or identifying) an inference execution layer utilizing the linear probing models, the perturbation autoencoder modeling system 106 can generate (or extract) cellular response representation embeddings from the particular inference execution layer of the machine learning model for input cellular response representations. Indeed, the perturbation autoencoder modeling system 106 can generate perturbation embeddings from input cellular response representations utilizing the inference execution layer as described in greater detail below (e.g., in reference to FIG. 6B).

As mentioned above, although conventional systems can utilize computer-based models to extract and analyze digital signals for images portraying cells, these conventional systems often have a number of problems in relation to computational efficiency, extraction accuracy, and flexibility of operation. For example, many conventional systems are computationally inefficient in training machine learning models to generate embeddings from microscopy images. Indeed, in many cases, conventional systems utilize classification-based models to extract and analyze digital signals for images portraying cells. In order to train such classification-based models, conventional systems often are required to perform segmentation and/or labeling of training images. In microscopy images, generating segmentations and/or training labels is difficult on a large number of training microscopy (or phenomic) images. Indeed, such segmentation and/or labeling is often time consuming, expensive, and computationally tasking. Furthermore, in many cases, a wide range of cellular phenotypes in images are difficult to interpret and/or annotate for segmentation and/or labeling.

As a result of the expense and limitations of creating training data via segmentation and/or classification labels, many conventional systems train classification-based models to extract and analyze digital signals for images portraying cells from smaller batches of training images. In order to achieve (or train) accurate classification-based machine learning models for microscopy image feature extraction from smaller batches of training data, many conventional systems repeatedly train the classification-based machine learning models using the smaller batches of training data. Oftentimes, conventional systems also repeat training for new classification and/or segmentation labels introduced in training images. Indeed, in many cases, training iterations with smaller batches of training data cause many conventional systems to repeat training multiple times and require thousands of graphical processing unit (GPU) hours for each training iteration. Accordingly, many conventional systems are often computationally and time-wise inefficient during training of machine learning models to generate embeddings from microscopy images.

In some cases, conventional systems attempt to utilize self-supervised learning approaches to train on larger training data sets (where labels are lacking or heavily biased). However, self-supervised learning approaches utilized by conventional systems are often inaccurate. For example, many conventional systems utilize self-supervised learning approaches that rely on augmentations inspired by natural images. However, augmentations inspired by natural images are often not applicable to many microscopy images (e.g., high-content screening (HCS) microscopy datasets).

Despite performing extensive and computationally expensive training, many conventional systems result in inaccurate machine learning models for microscopy image feature extraction. In particular, due to the limited samples in the smaller batches of training images, many conventional systems result in machine learning models that are not exposed to a wide variety of microscopy images (which limits the model's ability to perform accurate inferences). Indeed, conventional systems often utilize small batches of training data, due to the inefficiencies in generating segmentation and/or classification labels in training data, which result in under trained machine learning models.

Moreover, as a result of the inefficiencies and computational expenses of training approaches used by many conventional systems, these conventional systems are often inflexible in operation. For example, many conventional systems are unable to scale training to larger batches of training data because such training data requires segmentation and/or classification labeling. In addition, many conventional systems are also unable to easily adapt to new microscopy images (and/or new features depicted in the microscopy images) because the training may not have been performed on particular classifications or segmentations depicted in the new microscopy images.

Furthermore, some conventional systems utilize dataset curation on models. Indeed, some conventional systems often utilize pre-trained models for filtering and pruning, such as vision-language models to discard irrelevant pairs, semantic deduplication to remove redundancy, and prototypicality-based approaches to retain representative data. However, the above-mentioned techniques are less effective in high content screening system data (e.g., cellular response representations) where redundancy, variability, and subtle morphological differences make conventional filtering challenging.

As suggested by the foregoing, the perturbation autoencoder modeling system 106 provides a variety of technical advantages relative to conventional systems. For example, by utilizing a generative machine learning model (e.g., a masked autoencoder generative model) to generate phenomic (or other cellular response representation) embeddings from input phenomic images (or other cellular response representations), the perturbation autoencoder modeling system improves training efficiency. In particular, unlike conventional systems that utilize annotated training data, the perturbation autoencoder modeling system 106 can train a generative machine learning model to generate phenomic embeddings using a wide variety of phenomic images (e.g., without segmentation or classification labels). By utilizing a generative machine learning model to generate phenomic embeddings, the perturbation autoencoder modeling system 106 can utilize large training image batches (e.g., millions or billions of phenomic images as training data) to train the generative machine learning model.

In particular, unlike many conventional systems, the perturbation autoencoder modeling system 106 can utilize such large training image batches to reduce repetitive training for machine learning-based feature extraction from microscopy images (or other representations). Indeed, by being able to utilize larger training image batches, the perturbation autoencoder modeling system 106 can enable the generative machine learning model to learn from a wider variety of training images in less training iterations which reduces the amount of GPU hours utilized during training. Thus, the perturbation autoencoder modeling system 106 can efficiently train a machine learning model to extract features from microscopy images using a substantially larger amount of training phenomic images with an efficiency improvement in the computational resources and GPU time utilized to train the larger amount of training phenomic images.

In addition, the perturbation autoencoder modeling system 106 further improves the efficiency of utilizing generative machine learning models for feature extraction from cellular response representation embeddings. For example, the perturbation autoencoder modeling system 106 can filter training cellular response representation data utilizing perturbation significance identified from machine learning embeddings of the training cellular response representation data to generate a focused set of training cellular response representations to improve the efficiency of training the generative machine learning models (e.g., through the utilization of less training data) while improving the accuracy of the generative machine learning model. For example, by reducing noisy training data and utilizing a focused set of training cellular response representations that have perturbation significance to train the generative machine learning model, the perturbation autoencoder modeling system 106 improves the accuracy of extracted cellular response representation embeddings from cellular response representations. Moreover, the utilization of perturbation significance metrics improves the effective filtering of high content screening system data (e.g., cellular response representations) in contrast to conventional filtering approaches. Indeed, by utilizing filtering in accordance with one or more implementations herein, the perturbation autoencoder modeling system 106 improves the recall of known gene-gene relationships and consistency of embeddings for perturbations (e.g., gene knockout perturbations).

Moreover, the perturbation autoencoder modeling system 106 further improves the performance of the generative machine learning model by fine-tuning a subset of parameters of the generative machine learning model (trained for the cellular response representation completion task) utilizing a perturbation classification task. Indeed, the perturbation autoencoder modeling system 106 rebalances the parameters of the pre-trained generative machine learning model towards perturbation classification tasks to improve the accuracy of the output cellular response representation embeddings of the generative machine learning model.

In addition, the perturbation autoencoder modeling system 106 also improves the accuracy (or performance) of cellular response representation embedding extraction while reducing computational inference costs by utilizing linear probing models to select an intermediate layer of the generative machine learning model for embedding extraction. For instance, the perturbation autoencoder modeling system 106 utilizes linear probing to identify an intermediate layer that generates cellular response representation embeddings that generate accurate perturbation classifications (e.g., correlating to high performance embeddings). While improving the accuracy of the generative machine learning model via selection of a high accuracy inference execution layer, the perturbation autoencoder modeling system 106 also improves the efficiency of the generative machine learning model by reducing the number of iterations through identifying an intermediate layer of the generative machine learning model to extract the cellular response representation embeddings. In addition, by utilizing linear probing, the perturbation autoencoder modeling system evaluates output features of the intermediate layers with increased speed and efficiency compared to conventional evaluation approaches (such as biological relationship recall metrics) that are computationally expensive.

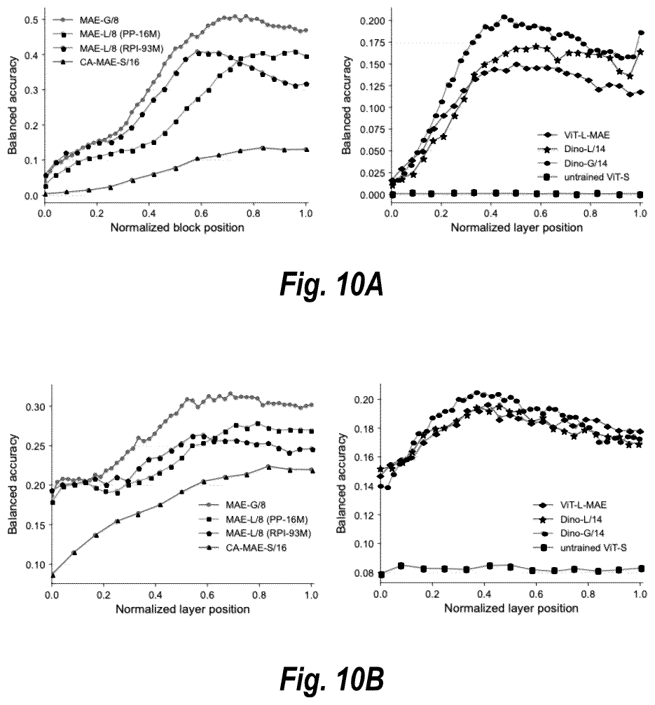

Indeed, experimental results illustrated with respect to FIGS. 10-13 demonstrate a variety of technical advantages and accuracy improvements provided by one or more implementations of the perturbation autoencoder modeling system 106.

As mentioned above, the perturbation autoencoder modeling system 106 trains and utilizes a generative machine learning model to generate cellular response representation embeddings from cellular response representations. For example, FIG. 3 illustrates an exemplary flow of the perturbation autoencoder modeling system 106 training a generative machine learning model to generate cellular response representation embeddings from cellular response representations (e.g., phenomic images using masked training phenomic images, transcriptomics representations using masked training transcriptomics representations). Additionally, FIG. 3 also illustrates an exemplary flow of the perturbation autoencoder modeling system 106 filtering training cellular response representation data utilizing perturbation significance identified from machine learning embeddings of the training cellular response representation data, utilizing a perturbation classification training task to fine-tune a subset of parameters of the generative machine learning model, and utilizing linear probing models to identify intermediate layers from the generative machine learning model to generate improved cellular response representation embeddings from a selected intermediate layer. Moreover, FIG. 3 also illustrates an exemplary flow of the perturbation autoencoder modeling system 106 generating cellular response representation autoencoder embeddings utilizing a trained generative machine learning model.

For example, as shown in FIG. 3, the perturbation autoencoder modeling system 106 identifies (or receives) training cellular response representations 306. Then, as shown in FIG. 3, the perturbation autoencoder modeling system 106 identifies (or generates) masked training cellular response representations 310 from the training cellular response representations 306 (and/or from the focused training cellular response representations 308). As shown in FIG. 3, the masked training cellular response representations 310 include training cellular response representations with masked patches and visible (or readable) patches from the training cellular response representations.

For instance, as used herein, the term “perturbation” (e.g., cell perturbation) refers to an alteration or disruption to a cell or the cell's environment (to elicit potential phenotypic changes to the cell). In particular, the term perturbation can include a gene perturbation (i.e., a gene-knockout perturbation) or a compound perturbation (e.g., a molecule perturbation or a soluble factor perturbation). These perturbations are accomplished by performing a perturbation experiment. A perturbation experiment refers to a process for applying a perturbation to a cell. A perturbation experiment also includes a process for developing/growing the perturbed cell into a resulting phenotype.

Thus, a gene perturbation can include gene-knockout perturbations (performed through a gene knockout experiment). For instance, a gene perturbation includes a gene-knockout in which a gene (or set of genes) is inactivated or suppressed in the cell (e.g., by CRISPR-Cas9 editing). The term perturbation can also include a small molecule perturbation (e.g., a compound perturbation), a protein perturbation, an antibody perturbation, a gene perturbation, a virus perturbation, or an in vivo perturbation.

Moreover, the term “compound perturbation” can include a cell perturbation using a molecule and/or soluble factor. For instance, a compound perturbation can include reagent profiling such as applying a small molecule to a cell and/or adding soluble factors to the cell environment. Additionally, a compound perturbation can include a cell perturbation utilizing the compound or soluble factor at a specified concentration. Indeed, compound perturbations performed with differing concentrations of the same molecule/soluble factor can constitute separate compound perturbations. A soluble factor perturbation is a compound perturbation that includes modifying the extracellular environment of a cell to include or exclude one or more soluble factors. Additionally, soluble factor perturbations can include exposing cells to soluble factors for a specified duration wherein perturbations using the same soluble factors for differing durations can constitute separate compound perturbations.

Moreover, as used herein, the term cellular response representation (or cellular response data) can refer to data that indicates or represents one or more characteristics of samples or other objects (e.g., cell structure samples, chemical objects, biological objects) obtained through microscopic instruments (e.g., a microscope, gene testing device). For example, a cellular response representation can include a phenomic image. Additionally, a cellular response representation can include transcriptomics data that indicates molecular structures expressed in a biological (or chemical) sample. For example, transcriptomics data can include an array or table of ribonucleic acid (RNA) or messenger RNA (mRNA) produced (e.g., an RNA count) in a cell or tissue sample for one or more perturbations. Indeed, a cellular response representation can include a variety of microscopy representations.

Furthermore, as used herein, the term phenomic image (or perturbation image), refers to a digital image portraying a cell (e.g., a cell after applying a perturbation). For example, a phenomic image includes a digital image of a stem cell after application of a perturbation and further development of the cell. Thus, a phenomic image comprises pixels that portray a modified cell phenotype resulting from a particular cell perturbation.

As also used herein, the term masked phenomic image refers to a phenomic image that is modified to conceal (or remove) one or more visible pixels depicted a cell phenotype from the phenomic image. For example, a masked phenomic image can include a phenomic image having a mask layer to create non-visible and visible patches in the phenomic image. For instance, the perturbation autoencoder modeling system 106 can modify a masked phenomic image by concealing (or removing) one or more visible pixels of a phenomic image with non-visible (e.g., blank or zeroed) pixels. In some instances, the perturbation autoencoder modeling system 106 can mask a phenomic image by introducing various amounts of patches to conceal (or remove) visible pixels of the phenomic image (e.g., 25%, 50%, 75%). In some instances, the perturbation autoencoder modeling system 106 utilizes random patches within a phonemic image to generate a masked image. In one or more implementations, the perturbation autoencoder modeling system 106 can also generate a masked phenomic image by introducing noise representations in a phenomic image (e.g., Gaussian noise, random noise, white noise).

As further shown in FIG. 3, the perturbation autoencoder modeling system 106 utilizes the masked training cellular response representations 310 with a generative machine learning model 312. Indeed, as shown in FIG. 3, during training, the perturbation autoencoder modeling system 106 utilizes the generative machine learning model 312 with the masked training cellular response representations 310 to generate (reconstructed) or predicted cellular response representations 314. For instance, as illustrated in FIG. 3, the perturbation autoencoder modeling system 106 utilizes the generative machine learning model 312 to reconstruct the masked training cellular response representations 310 into representations of the training cellular response representations 306 (and/or the focused training cellular response representations 308) as the predicted cellular response representations 314.

Moreover, as shown in FIG. 3, the perturbation autoencoder modeling system 106 determines a measure of loss 316 for the generative machine learning model 312 by comparing the predicted cellular response representations 314 with the training cellular response representations 306 (and/or the focused training cellular response representations 308). In particular, the perturbation autoencoder modeling system 106 compares the predicted cellular response representations 314 to the training cellular response representations 306 (and/or the focused training cellular response representations 308) to determine the measure of loss 316 to quantify errors (or inaccuracies) between the reconstrued predicted cellular response representations 314 and the original (ground truth) training cellular response representations 306 (and/or the focused training cellular response representations 308). Indeed, as illustrated in FIG. 3, the perturbation autoencoder modeling system 106 utilizes the measure of loss 316 with the generative machine learning model 312 to modify parameters of the generative machine learning model 312. For example, the perturbation autoencoder modeling system 106 can iteratively generate predicted cellular response representations 314 from the masked training cellular response representations 310 using the generative machine learning model 312 with modified parameters to reduce (or minimize) the measure of loss 316 between the predicted cellular response representations 314 and the training cellular response representations 306 (and/or the focused training cellular response representations 308).

In addition, as shown in FIG. 3, upon training the generative machine learning model 312, the perturbation autoencoder modeling system 106 can utilize the generative machine learning model 312 to generate cellular response representation embeddings. Indeed, as shown in FIG. 3, the perturbation autoencoder modeling system 106 can identify (or receive) a cellular response representation(s) 318. Moreover, as shown in FIG. 3, the perturbation autoencoder modeling system 106 can utilize the cellular response representation(s) 318 with the generative machine learning model 312 (via an encoder) to generate cellular response representation autoencoder embeddings 324.

As used herein, the term “machine learning model” includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques (e.g., supervised or unsupervised learning) to improve in accuracy and/or effectiveness. Example machine learning models include various types of decision trees, support vector machines, Bayesian networks, random forest models, or neural networks (e.g., deep neural networks, generative adversarial neural networks, convolutional neural networks, recurrent neural networks, or diffusion neural networks). Similarly, the term “machine learning data” refers to information, data, or files generated or utilized by a machine learning model. Machine learning data can include training data, machine learning parameters, or embeddings/predictions generated by a machine learning model.

As also used herein, the term “generative machine learning model” refers to a deep learning model that generates a digital image or another digital representation (e.g., from a masked representation such as a noisy image, a randomly masked image, masked tabular data). For example, the generative machine learning model can include a deep learning model that is trained to reverse noise introduced in a training image to reconstruct the training image (e.g., trained to remove masks or noise to generate a representation of the training image). For example, the perturbation autoencoder modeling system 106 can train a generative machine learning model to iteratively denoise a masked version of a training phenomic image to generate a reconstructed (i.e., predicted) version of the training phenomic image. Moreover, in some cases, during inference, the perturbation autoencoder modeling system 106 can utilize the trained generative machine learning model to generate perturbation embeddings for an input phenomic image. In some instances, the perturbation autoencoder modeling system 106 can train a generative machine learning model to denoise a masked cellular response representation (e.g., a masked transcriptomics representation) to generate a reconstructed version of a training cellular response representation (e.g., to utilize the trained model to generate cellular response representation embeddings from input cellular response representations).

In some instances, the perturbation autoencoder modeling system 106 utilizes a masked autoencoder machine learning model (or sometimes referred to as “masked autoencoder”) as the generative machine learning model. For example, a masked autoencoder machine learning model can, during training, utilize an encoder-decoder architecture that encodes a subset of visible image patches from a masked phenomic image and utilizes a decoder to reconstruct an original phenomic image (of the masked phenomic image) or other cellular response representation (as described below). Furthermore, after training, the perturbation autoencoder modeling system 106 can utilize the encoder of masked autoencoder machine learning model on phenomic images to generate perturbation embeddings that are utilized in various recognition and/or analysis tasks. For example, the perturbation autoencoder modeling system 106 can train and utilize a masked autoencoder as described in Kaiming He et al., Masked Autoencoders Are Scalable Vision Learners, arXiv, arXiv:211.06377v3 (2021) (hereinafter “He”), which is incorporated herein by reference in its entirety.

For instance, in some implementations, the perturbation autoencoder modeling system 106 can embed phenomic images into a low dimensional feature space via a generative machine learning model (e.g., a masked autoencoder model or channel-agnostic masked autoencoder model) to generate perturbation image embeddings (or phenomic perturbation autoencoder embeddings). As used herein, the term “perturbation embedding” (or perturbation autoencoder embeddings, phenomic perturbation autoencoder embeddings, cellular response representation embeddings, or phenomic image embeddings) refers to a numerical representation of a phenomic image. For example, a perturbation embedding includes a vector representation of a phenomic image generated by a machine learning model (e.g., a masked autoencoder generative model in accordance with one or more embodiments herein). Thus, a perturbation embedding includes a feature vector generated by application of various machine learning (or encoder) layers (at different resolutions/dimensionality).

In some instances, the perturbation autoencoder modeling system 106 can embed other cellular response representations (e.g., transcriptomics representations) into a low dimensional feature space via a generative machine learning model to generate cellular response representation (or perturbation) embeddings (e.g., a numerical and/or feature vector representation of transcriptomics data). For instance, a cellular response representation embedding can include a vector representation of transcriptomics data generated by a machine learning model.

Indeed, FIG. 3 illustrates the perturbation autoencoder modeling system 106 training and utilizing a generative machine learning model with various types of cellular response representations. As shown in FIG. 3, in some cases, the perturbation autoencoder modeling system 106 can train and utilize a generative machine learning model with phenomic images. For instance, as shown in FIG. 3, the perturbation autoencoder modeling system 106 can utilize training phenomic images and masked training phenomic images to train the generative machine learning model to reconstruct the masked training phenomic images into versions of the training phenomic images. In addition, the perturbation autoencoder modeling system 106 can utilize the trained generative machine learning model with input phenomic images to generate phenomic perturbation autoencoder embeddings for the input phenomic images.

In addition to or as an alternative embodiment, the perturbation autoencoder modeling system 106 can train and utilize a generative machine learning model with other cellular response representations (e.g., transcriptomics data). For instance, as shown in FIG. 3, the perturbation autoencoder modeling system 106 can train and utilize a generative machine learning model with transcriptomics data. As an example, the perturbation autoencoder modeling system 106 can receive training transcriptomics data that indicate a number of RNA counts expressed for one or more perturbations (within a particular gene). For instance, the training transcriptomics data can include an array or table of RNA count data for a number of perturbations (e.g., in rows of the table) corresponding to particular genes (e.g., in columns of the table).

Moreover, the perturbation autoencoder modeling system 106 can generate masked training cellular response representations from the training transcriptomics representations (or data) by masking (or hiding) one or more elements (or entries) in the transcriptomics data. For instance, the perturbation autoencoder modeling system 106 can delete or remove one or more RNA count entries in the transcriptomics representations to generate masked training cellular response representations. Furthermore, the perturbation autoencoder modeling system 106 can utilize the training transcriptomics representations and masked training transcriptomics representations to train the generative machine learning model to reconstruct the masked training transcriptomics representations into versions of the training transcriptomics representations (e.g., by filling in missing RNA counts in the array or table). In addition, the perturbation autoencoder modeling system 106 can utilize the trained generative machine learning model with input transcriptomics data to transcriptomics data autoencoder embeddings for the input transcriptomics data. Indeed, the perturbation autoencoder modeling system 106 can utilize the transcriptomics data autoencoder embeddings to compare different transcriptomics data instances (e.g., compare multiple transcriptomics arrays) in accordance with one or more implementations herein.

For example, the perturbation autoencoder modeling system 106 can train and utilize a generative machine learning model to generate cellular response representation embeddings from cellular response representations as described in UTILIZING MASKED AUTOENCODER GENERATIVE MODELS TO EXTRACT MICROSCOPY REPRESENTATION AUTOENCODER EMBEDDINGS, U.S. patent application Ser. No. 18/545,399, filed Dec. 19, 2023 (hereinafter U.S. patent application Ser. No. 18/545,399), which is incorporated herein by reference in its entirety. Furthermore, the perturbation autoencoder modeling system 106 can train and utilize a generative machine learning model with cellular response representations (e.g., phenomic images, transcriptomics data) utilizing momentum-training optimizers, Fourier transformation losses, multi-weighted losses, and/or channel agnostic training as described in U.S. patent application Ser. No. 18/545,399. Indeed, the perturbation autoencoder modeling system 106 can utilize a generative machine learning model as described in U.S. patent application Ser. No. 18/545,399 with one or more implementations of the perturbation autoencoder modeling system 106 described herein.

As mentioned above, the perturbation autoencoder modeling system 106 can further improve the performance of generating accurate perturbation embeddings from the generative machine learning model. For example, FIG. 3 also illustrates an exemplary flow of the perturbation autoencoder modeling system 106 training a generative machine learning model to generate cellular response representation embeddings utilizing filtration of training cellular response representation data utilizing perturbation significance and/or fine-tuning a subset of parameters of the generative machine learning model (trained for the cellular response representation completion task) utilizing a perturbation classification task. Furthermore, FIG. 3 also illustrates an exemplary flow of the perturbation autoencoder modeling system 106 utilizing linear probing models to identify intermediate layers from the generative machine learning model to generate accurate cellular response representation embeddings from a selected intermediate layer.

For example, as shown in an act 302 of FIG. 3, the perturbation autoencoder modeling system 106 filters training cellular response representations. In particular, the perturbation autoencoder modeling system 106 determines perturbation significance metrics 304 for the training cellular response representations 306 (e.g., utilizing embeddings of the training cellular response representations 306) that indicate a noticeability of expression (e.g., a visual expression or a gene count expression) from the training cellular response representations 306. Furthermore, the perturbation autoencoder modeling system 106 can utilize the perturbation significance metrics 304 to filter the training cellular response representations 306 to generate (or determine) the focused training cellular response representations 308.

As used herein, the term “perturbation significance metric” refers to a measure that represents the significance or noticeability of a phenoprint (or morphological change) in a cellular response representation. In particular, the perturbation significance metric can include a statistical significance through a compared measure between a consistency of an induced morphology on cells by a perturbation (e.g., a perturbation consistency value) and a null distribution of sampled perturbation consistency values from other perturbations (e.g., randomly selected perturbations). Moreover, as used herein, the term “perturbation consistency value” refers to a measure of consistency of a cellular response representation (or cellular response representation embedding) representing an induced morphology on one or more cells by a perturbation through similarity measures with replicate cellular response representations of the particular perturbation. Indeed, a perturbation consistency value can include a mean of cosine similarities across all pairs of replicates (e.g., perturbation embeddings) of a particular perturbation.

In addition, as also shown in FIG. 3, the perturbation autoencoder modeling system 106 can fine-tune a subset of parameters of the generative machine learning model (trained for the cellular response representation completion task) utilizing a perturbation classification task. For instance, as shown in FIG. 3, the perturbation autoencoder modeling system 106 can utilize the generative machine learning model 312 to generate predicted perturbation classifications 326 for the focused training cellular response representations 308 (or the training cellular response representations 306). Moreover, as shown in FIG. 3, the perturbation autoencoder modeling system 106 can compare the predicted perturbation classifications 326 to ground truth perturbation classification data corresponding to the focused training cellular response representations 308 to generate the measure of loss 316. In addition, the perturbation autoencoder modeling system 106 can utilize the measure of loss 316 generated from the predicted perturbation classifications 326 to fine tune or modify a subset of parameters (e.g., unfrozen layer norms) of the generative machine learning model 312 (while freezing one or more layers or parameters of the generative machine learning model 312 upon training for the cellular response representation completion task via the predicted cellular response representations 314).

As used herein, the term “perturbation classification” (sometimes referred to as “perturbation classification prediction”) refers to a determined perturbation label indicating the perturbation associated with a cellular response representation (e.g., a phenomic image, a transcriptomic profile). In particular, a perturbation classification can include a label indicating a particular gene knockout perturbation and/or compound perturbation applied to a cell represented in the cellular response representation to induce the morphological change depicted (or demonstrated) in the cellular response representation. For instance, perturbation classifications can include perturbation class predictions generated utilizing a machine learning classifier model with input cellular response representations.

Moreover, as shown in an act 320 of FIG. 3, the perturbation autoencoder modeling system 106 can select an inference execution layer of the generative machine learning model. In particular, as shown in FIG. 3, the perturbation autoencoder modeling system 106 can utilize linear probing model(s) 322 with feature outputs (e.g., cellular response representation embeddings) from intermediate layers of the generative machine learning model 312 to select an inference execution layer for the generative machine learning model 312. Indeed, the perturbation autoencoder modeling system 106 can utilize linear probing models trained to generate perturbation classifications from cellular response representation embeddings to generate perturbation classifications from different sets of cellular response representation embeddings extracted from intermediate layers of generative machine learning model 312.

Furthermore, the perturbation autoencoder modeling system 106 can utilize the perturbation classifications corresponding to each set of cellular response representation embeddings to determine a classification accuracy of the particular set of cellular response representation embeddings from the perturbation classifications at each intermediate layer. In addition, the perturbation autoencoder modeling system 106 can utilize the determined classification accuracies to identify (or select) a layer of the generative machine learning model 312 as the inference execution layer. In one or more embodiments, the perturbation autoencoder modeling system 106 utilizes the determined inference execution layer of the generative machine learning model 312 to generate cellular response representation autoencoder embeddings 324 from the cellular response representation(s) 318.

As used herein, the term “intermediate layer” refers to an encoder block of a machine learning model or a set of parameters of the machine learning model. For instance, the perturbation autoencoder modeling system 106 can utilize, as an intermediate layer, one or more transformer blocks of a generative machine learning model (e.g., a vision transformer block). In addition, as used herein, the term “inference execution layer” refers to a particular encoder block or set of parameters of the machine learning model utilized to extract cellular response representation embeddings from the generative machine learning model. For instance, the inference execution layer can include an Nth transformer block (or layer) at which the perturbation autoencoder modeling system 106 extracts cellular response representation embeddings from the generative machine learning model (e.g., stopping or terminating further iterations or time steps of the generative machine learning model).

As used herein, the term “linear probing model” (sometimes referred to as “block-wise linear probing model”) refers to a model trained to generate perturbation classification predictions from output features (e.g., cellular response representation embeddings) derived from various intermediate layers of the generative machine learning model. In particular, the linear probing model can include a block-wise logistic regression model. For instance, the linear probing models can include a block-wise logistic regression models trained on output features of each transformer blocks of the generative machine learning model to predict a perturbation (e.g., the gene that was perturbed or the functional group that the gene belongs to).

In addition, as used herein, the term “perturbation classification accuracy metric” refers to a measure indicating an accuracy of correct perturbation classification predictions generated a particular set of cellular response representation embeddings. For instance, the perturbation classification accuracy metric can include a test balanced accuracy from perturbation classification predictions generated via a linear probing model from a set of cellular response representation embeddings.

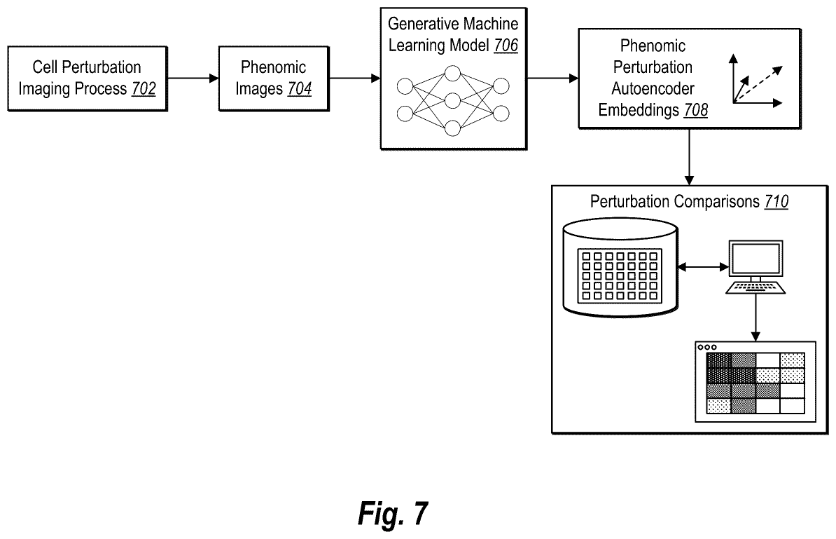

Furthermore, FIGS. 4-9 illustrate one or more embodiments of the perturbation autoencoder modeling system 106 training and utilizing a generative machine learning model with phenomic images. Although, FIGS. 4-9 illustrate embodiments of the perturbation autoencoder modeling system 106 for phenomic images, the perturbation autoencoder modeling system 106 can train and utilize a generative machine learning model with other cellular response representations (e.g., images, transcriptomics data) in accordance with one or more implementations of FIGS. 4-9. Indeed, the perturbation autoencoder modeling system 106 can implement the embodiments described in FIGS. 4-9 with various cellular response representations (e.g., phenomic images, transcriptomics data). For example, the perturbation autoencoder modeling system 106 can train and utilize a generative machine learning model with other cellular response representations (e.g., images, transcriptomics data) utilizing perturbation significance training data filtering, training a subset of layers (or parameters) using a perturbation classification prediction task, and/or utilizing inference execution layer selection as described in FIGS. 4-9. Moreover, the perturbation autoencoder modeling system 106 can utilize various types of cellular response representation embeddings to generate perturbation comparisons and/or cellular response representation corrections (as described in FIGS. 7-9).

As mentioned above, the perturbation autoencoder modeling system 106 can filter training cellular response representation data based on perturbation significance identified from machine learning embeddings of the training cellular response representation data to generate a focused set of training cellular response representations. Indeed, FIG. 4 illustrates the perturbation autoencoder modeling system 106 filtering training cellular response representation data utilizing perturbation significance identified from machine learning embeddings of the training cellular response representation data to generate a focused set of training cellular response representations for training of a generative machine learning model.

As shown in FIG. 4, the perturbation autoencoder modeling system 106 utilizes a machine learning model(s) 404 with training cellular response representations 402 to generate cellular response representation embeddings 406. Additionally, as shown in FIG. 4, the perturbation autoencoder modeling system 106 determines perturbation significance values for each embedding from the cellular response representation embeddings 406 (e.g., embeddings 410a-410n). In particular, as shown in FIG. 4, the perturbation autoencoder modeling system 106 (as part of a filtration model 408) compares an embedding 410a to a subset of embedding(s) 412a (e.g., embeddings from replicate cellular response representations of a perturbation) to determine a perturbation consistency value 413a (e.g., a similarity measure). Moreover, as shown in FIG. 4, the perturbation autoencoder modeling system 106 compares the perturbation consistency value 413a to a null distribution of perturbation consistency values 414 to generate the perturbation significance value 416a. Indeed, the perturbation autoencoder modeling system 106 can generate perturbation significance values 416b-416n from comparisons between perturbation consistency values 413b-413n (of embeddings 410b-410n and subset of embedding(s) 412b-412n) with the null distribution of perturbation consistency values 414 (in accordance with one or more implementations herein).

Furthermore, as shown in FIG. 4, the perturbation autoencoder modeling system 106 can, as part of the filtration model 408, filter the cellular response representation embeddings 406 to determine a focused subset of training cellular response representations 420 utilizing the perturbation significance values 416a-416n for the embeddings 410a-410n (e.g., the cellular response representation embeddings 406). In particular, as shown in FIG. 4, the perturbation autoencoder modeling system 106 compares the perturbation significance values 416a-416n to a threshold perturbation significance value 418 to identify embeddings from the embeddings 410a-410n corresponding to perturbation significance values that satisfy the threshold perturbation significance value 418. Indeed, the perturbation autoencoder modeling system 106 can identify the cellular response representations corresponding to the embeddings associated with the perturbation significance values that satisfy the threshold perturbation significance value 418 as the focused subset of training cellular response representations 420. Moreover, as shown in FIG. 4, the perturbation autoencoder modeling system 106 utilizes the focused subset of training cellular response representations 420 to train one or more parameters of a machine learning model 422.

In one or more instances, the perturbation autoencoder modeling system 106 utilizes perturbation consistency values to filter training cellular response representations to generate a balanced dataset of training cellular response representation data across semantic classes (e.g., to improve the effective learning under masked objectives of the generative machine learning model). For example, the perturbation autoencoder modeling system 106 can filter cellular response representations that look like unperturbed cells which are often over-represented because many perturbations may not often induce morphological changes to cells. Accordingly, the perturbation autoencoder modeling system 106 can utilize a perturbation consistency model (e.g., a non-parametric perturbation consistency model) that generates perturbation consistency values for the focused subset of training cellular response representations from initial latent representations of the training cellular response representations. Moreover, the perturbation autoencoder modeling system 106 can generate perturbation significance values by comparing the perturbation consistency values to a null distribution of perturbation consistency values determines from a sampled set of the training cellular response representations. Indeed, the perturbation autoencoder modeling system 106 can filter the training cellular response representations utilizing the perturbation significance values.

In one or more implementations, the perturbation autoencoder modeling system 106 can utilize a machine learning model to generate initial latent representations for the training cellular response representations. For instance, the perturbation autoencoder modeling system 106 can utilize a generative machine learning model (such as an autoencoder generative machine learning model) as described in U.S. patent application Ser. No. 18/545,399 to generate the initial latent representations for the training cellular response representations. For example, in one or more implementations, the perturbation autoencoder modeling system 106 can utilize a generative machine learning model trained to generate cellular response representation embeddings from cellular response representations utilizing momentum-training optimizers, Fourier transformation losses, and/or multi-weighted losses (as described in U.S. patent application Ser. No. 18/545,399) to generate the initial latent representations for the training cellular response representations.

In one or more embodiments, the perturbation autoencoder modeling system 106 can utilize a generative machine learning model trained in accordance with one or more implementations herein. For example, the perturbation autoencoder modeling system 106 can utilize a generative machine learning model to generate initial latent representations for the training cellular response representations. Subsequently, the perturbation autoencoder modeling system 106 can utilize the initial latent representations for the training cellular response representations to filter one or more training cellular response representations to generate the focused subset of training cellular response representations. Moreover, the perturbation autoencoder modeling system 106 can utilize the focused subset of training cellular response representations (determined from utilizing the generative machine learning model) to further train the generative machine learning model to generate accurate cellular response representation embeddings in accordance with one or more implementations herein.

In some instances, the perturbation autoencoder modeling system 106 can generate the initial latent representations utilizing a classification prediction machine learning model. As an example, the perturbation autoencoder modeling system 106 can generate cellular response representation embeddings from a supervised deep image embedding model. To illustrate, the perturbation autoencoder modeling system 106 can apply a supervised deep image embedding model (e.g., via a convolutional neural network model) to a phenomic image of a cell to generate a phenomic image embedding (e.g., a cellular response representation embedding). For example, the perturbation autoencoder modeling system 106 trains the supervised deep image embedding model to generate predicted perturbations from phenomic digital images. Indeed, the perturbation autoencoder modeling system 106 utilizes neural network layers to generate vector representations of the phenomic digital images at different levels of abstraction and then utilize output layers to generate predicted perturbations. The perturbation autoencoder modeling system 106 then trains the supervised deep image embedding model by comparing the predicted perturbations with ground truth perturbations. Moreover, the perturbation autoencoder modeling system 106 can utilize the internal feature vectors generated by the supervised deep image embedding model (for an input phenomic image) as the phenomic image embeddings (e.g., cellular response representation embeddings).

In some cases, the perturbation autoencoder modeling system 106 can generate the initial latent representations utilizing multiple machine learning models. For example, the perturbation autoencoder modeling system 106 can generate, as part of the initial latent representations, a first set of initial latent representations from a first machine learning model and a second set of initial latent representations from a second machine learning model. In some implementations, the perturbation autoencoder modeling system 106 combines embeddings from multiple machine learning models. Moreover, the perturbation autoencoder modeling system 106 can identify the focused subset of training cellular response representations by filtering (in accordance with one or more implementations herein) the initial latent representations from the first set of initial latent representations and the second set of initial latent representations.

Furthermore, as mentioned above, the perturbation autoencoder modeling system 106 can utilize a perturbation consistency model (e.g., a non-parametric perturbation consistency model) to generate perturbation consistency values from the initial latent representations of the training cellular response representations. As an example, the perturbation autoencoder modeling system 106 can determine similarity measures between a training cellular response representation embedding corresponding to a particular perturbation (e.g., the initial latent representations of the training cellular response representations) and a subset of additional training cellular response representation embeddings corresponding to the same particular perturbation. In addition, the perturbation autoencoder modeling system 106 can generate the perturbation consistency value for the training cellular response representation by combining the similarity measures between the training cellular response representation embedding and the subset of additional training cellular response representation embeddings corresponding to the same particular perturbation.

In some cases, the perturbation autoencoder modeling system 106 can generate the perturbation consistency values as described in Safiye Celik et. al., Building, Benchmarking, and Exploring Perturbative Maps of Transcriptional and Morphological Data, PLOS Computational Biology, 20(10): 1-24, (2024), which is incorporated herein by reference in its entirety.

In one or more cases, the perturbation autoencoder modeling system 106 can utilize a variety of similarity measures. For example, the perturbation autoencoder modeling system 106 can utilize similarity measures, such as, but not limited to, cosine similarities and/or Euclidean distances. Additionally, in one or more embodiments, the perturbation autoencoder modeling system 106 can combine similarity measures to generate the perturbation consistency value. For instance, the perturbation autoencoder modeling system 106 can combine the similarity measures by utilizing a mean similarity measure (e.g., a mean cosine similarity) between the training cellular response representation embedding and the subset of additional training cellular response representation embeddings corresponding to the same particular perturbation. In some cases, the perturbation autoencoder modeling system 106 can combine the similarity measures utilizing a variety of approaches, such as, but not limited to, a summation of similarity measures and/or a median of similarity measures.

As an example, in one or more implementations, the perturbation autoencoder modeling system 106 can determine the consistency of an induced morphology on cells by perturbations (e.g., perturbation significance values for cellular response representations) utilizing a non-parametric perturbation consistency test. In particular, the perturbation autoencoder modeling system 106 can identify cellular response representation embeddings x_{g,1}, x_{g,2}, . . . , x_{g,n} of replicates of a perturbation x_{g} from an experiment batch e. In addition, the perturbation autoencoder modeling system 106 can generate a perturbation consistency value as the mean of the cosine similarities

$$( {\overline{s}}_{g}^{e} )$$

across all pairs of replicates of x_{g} in accordance with the following function:

$${\overline{s}}_{g}^{e} = \frac{1}{{n}^{2}} {\sum }_{i = 1}^{n} {\sum }_{j = 1}^{n} \frac{〈 {x}_{g , i} , {x}_{g , j} 〉}{‖ {x}_{g , i} ‖ ‖ {x}_{g , j} ‖} \qquad ( 1 )$$

For instance, in reference to function (1) above, the perturbation autoencoder modeling system 106 can utilize a dot product [symbol] ⋅ [symbol] and an L₂ norm (∥⋅∥) to generate the perturbation consistency value as the mean of the cosine similarities

$$( {\overline{s}}_{g}^{e} ) .$$

In addition, the perturbation autoencoder modeling system 106 can utilize the perturbation consistency value to generate a perturbation significance value for the particular training cellular response representation. In particular, the perturbation autoencoder modeling system 106 can compare the perturbation consistency value

$$( e . g . , {s}_{g}^{- e} )$$

against an empirical null distribution generated using perturbation consistency values for a set of randomly sampled (or selected) perturbations (e.g., in an experiment e) as perturbation consistency values

$${\overline{s}}_{1}^{′} , ... , {\overline{s}}_{K}^{′} .$$

In some instances, the perturbation autoencoder modeling system 106. Indeed, the perturbation autoencoder modeling system 106 can generate the perturbation significance value (e.g., a p-value p_{g}) for the between the randomly sampled perturbation consistency values

$${\overline{s}}_{k}^{′}$$

and the perturbation consistency value

$${\overline{s}}_{g}^{e}$$

of a particular perturbation in accordance with the following function:

$${p}_{g} = \frac{max \{ {\overline{s}}_{k}^{′} \leq {\overline{s}}_{g}^{e} \} , 1}{K} \qquad ( 2 )$$

In some cases, the perturbation autoencoder modeling system 106 can generate a perturbation significance value for a cellular response representation (or perturbation corresponding to the cellular response representation) across multiple perturbation experiment data sets. For instance, the perturbation autoencoder modeling system 106 can combine perturbation significance values (e.g., p-values) from the multiple perturbation experiment data sets for a perturbation utilizing a Cauchy Combination test as described in Yaowu Liu et. al., Cauchy Combination Test: A Powerful Test with Analytic P-Value Calculation Under Arbitrary Dependency Structures, Journal of the American Statistical Association, 115:393-402 (2018), which is incorporated herein by reference in its entirety. In one or more instances, the perturbation autoencoder modeling system 106 can combine perturbation significance values (e.g., p-values) from the multiple perturbation experiment data sets utilizing a variety of approaches, such as, generating a mean perturbation significance value from multiple perturbation significance values and/or generating a median perturbation significance value from multiple perturbation significance values.

Furthermore, the perturbation autoencoder modeling system 106 can filter (or select) a training cellular response representation for the focused subset of training cellular response representations by comparing a perturbation significance value corresponding to a training cellular response representation to a threshold perturbation significance value. For example, the perturbation autoencoder modeling system 106 can determine whether the perturbation significance value corresponding satisfies the threshold perturbation significance value (e.g., is less than or equal to the threshold p-value, is less than the threshold p-value or vice versa). Moreover, the perturbation autoencoder modeling system 106 can configure the threshold perturbation significance value based on user inputs from an administrator device. Moreover, the perturbation autoencoder modeling system 106 can utilize a variety of threshold perturbation significance values (e.g., 0.008, 0.01, 0.02, 0.05). Upon identifying a training cellular response representation with a perturbation significance value that satisfies the threshold perturbation significance value, the perturbation autoencoder modeling system 106 adds the training cellular response representation to the focused subset of training cellular response representations.

In addition, the perturbation autoencoder modeling system 106 can iteratively (or repeatedly) filter training cellular response representations to generate the focused subset of training cellular response representations. For instance, the perturbation autoencoder modeling system 106 can utilize filtered training cellular response representations with an additional machine learning model to generate additional training cellular response representations. The perturbation autoencoder modeling system 106 can further filter the additional training cellular response representations by determine perturbation significance values for the additional training cellular response representations. For example, the perturbation autoencoder modeling system 106 can utilize a generative machine learning model (as described above), an image classification model (as described above), and/or a weakly supervised learning model.

Moreover, the perturbation autoencoder modeling system 106 can further filter the training cellular response representations utilizing a variety of filtering approaches. For example, the perturbation autoencoder modeling system 106 can filter the training cellular response representations based on cellular response representation data quality (e.g., a data quality filter related to the focus of the image, quantity of dead cells, assay conditions, and/or presence of strong anomalous imaging artifacts). In some cases, the perturbation autoencoder modeling system 106 can filter the training cellular response representations utilizing data discrepancies (e.g., a cellular response representation with missing information, data with excessive or multiple perturbation applications, and/or data of unusual size in terms of resolution, dimension, and/or channels). Furthermore, the perturbation autoencoder modeling system 106 can filter the training cellular response representations utilizing perturbation conditions associated with the training cellular response representations (e.g., perturbation conditions that are present in less than a threshold number of distinct experiments and/or a threshold number of distinct wells to capture a variety of batch effects and broaden samples of positives per class). Additionally, the perturbation autoencoder modeling system 106 can filter the training cellular response representations by under-sampling one or more perturbation conditions (e.g., under-sampling over-represented perturbation conditions based on a threshold amount of data per perturbation condition). In some cases, the perturbation autoencoder modeling system 106 can under-sample by utilizing proportions between different experiment control data (e.g., a particular percentage of positive controls, negative controls, and/or wells without perturbation within each experiment).

In some instances, the perturbation autoencoder modeling system 106 can utilize the perturbation significance values determined for the training cellular response representations to reweight training cellular response representations. For instance, the perturbation autoencoder modeling system 106 can utilize the perturbation significance values to assign weights to each of the training cellular response representations. Moreover, the perturbation autoencoder modeling system 106 can train the machine learning model (in accordance with one or more implementations herein) utilizing the training cellular response representations weighted according to the perturbation significance values of the training cellular response representations.

As mentioned above, the perturbation autoencoder modeling system 106 can train a machine learning model utilizing both a cellular response representation completion task and a perturbation classification task. In particular, FIGS. 5A and 5B illustrate the perturbation autoencoder modeling system 106 training a machine learning model utilizing both a cellular response representation completion task and a perturbation classification task (with focused training cellular response representations). In particular, FIGS. 5A and 5B illustrate the perturbation autoencoder modeling system 106 training the machine learning model (e.g., a generative machine learning model) on focused training cellular response representations via a cellular response representation completion task and subsequently training a subset of layers (or parameters) of the generative machine learning model via a perturbation classification task.

For instance, as shown in FIG. 5A, the perturbation autoencoder modeling system 106 identifies (or generates) masked training cellular response representations 504 from focused training cellular response representations 502. Moreover, as shown in FIG. 5A, utilizes the masked training cellular response representations 504 with a machine learning model 506. Indeed, as shown in FIG. 5A, during training, the perturbation autoencoder modeling system 106 utilizes the machine learning model 506 with the masked training cellular response representations 504 to generate (reconstructed) or predicted cellular response representations 508. For instance, as illustrated in FIG. 5A, the perturbation autoencoder modeling system 106 utilizes the machine learning model 506 to reconstruct the masked training cellular response representations 504 into representations of the focused training cellular response representations 502 as the predicted cellular response representations 508.

Moreover, as shown in FIG. 5A, the perturbation autoencoder modeling system 106 determines a measure of loss 510 for the machine learning model 506 by comparing the predicted cellular response representations 508 with the focused training cellular response representations 502. For instance, the perturbation autoencoder modeling system 106 compares the predicted cellular response representations 508 to the focused training cellular response representations 502 to determine the measure of loss 510 to quantify errors (or inaccuracies) between the reconstrued predicted cellular response representations 508 and the original (ground truth) focused training cellular response representations 502. Indeed, as illustrated in FIG. 5A, the perturbation autoencoder modeling system 106 utilizes the measure of loss 510 with the machine learning model 506 to modify parameters of various layers (e.g., layers 1-N) of the machine learning model 506. For example, the perturbation autoencoder modeling system 106 can iteratively generate predicted cellular response representations 508 from the masked training cellular response representations 504 using the machine learning model 506 with modified parameters to reduce (or minimize) the measure of loss 510 between the predicted cellular response representations 508 and the focused training cellular response representations 502.

Furthermore, as mentioned above, the perturbation autoencoder modeling system 106 can freeze layers (or parameters) of the machine learning model 506 upon training the machine learning model 506 via the cellular response representation completion task and subsequently train a subset of unfrozen layers (or parameters) of the machine learning model 506 via a perturbation classification prediction task. Indeed, FIG. 5B illustrates the perturbation autoencoder modeling system 106 subsequently training a subset of unfrozen layers (or parameters) of the machine learning model 506 via a perturbation classification prediction task.

For instance, as shown in FIG. 5B, the perturbation autoencoder modeling system 106 can utilize the machine learning model 506 with the focused training cellular response representations 502 (as input) to generate predicted perturbation classifications 512. In one or more instances, the perturbation autoencoder modeling system 106 utilizes the machine learning model 506 to generate the predicted perturbation classifications 512 to indicate a predicted perturbation utilized (or corresponding with) the input focused training cellular response representations 502.

In addition, as shown in FIG. 5B, the perturbation autoencoder modeling system 106 can determine a measure of loss 514 for the machine learning model 506 by comparing the predicted perturbation classifications 512 with ground truth perturbation classifications 516 of the focused training cellular response representations 502 (e.g., ground truth perturbation classification labels corresponding to the focused training cellular response representations 502). In particular, the perturbation autoencoder modeling system 106 compares the predicted perturbation classifications 512 to the ground truth perturbation classifications 516 to determine the measure of loss 514 to quantify errors (or inaccuracies) between the predicted perturbation classifications 512 and the ground truth perturbation classifications 516.

Furthermore, the perturbation autoencoder modeling system 106 utilizes the measure of loss 514 to modify (or train) select layers or parameters of the machine learning model 506 (having frozen layers from pre-training on a cellular response representation completion task). For instance, as shown in FIG. 5B, the perturbation autoencoder modeling system 106 utilizes the measure of loss 514 to modify parameters of layer 2 (e.g., a layer norm) of the machine learning model 506 without affecting or modifying parameters of other layers of the machine learning model 506. Indeed, the perturbation autoencoder modeling system 106 can utilize the measure of loss 514 (from the predicted perturbation classification task) to modify a variety of parameters or layers of the machine learning model 506.

For example, the perturbation autoencoder modeling system 106 can freeze a set of layers (e.g., a first set of layers) for a pre-trained machine learning model (e.g., trained via a cellular response representation task in accordance with one or more implementations herein). Subsequently, the perturbation autoencoder modeling system 106 can modify a second set of layers (e.g., unfrozen layers) utilizing a measure of loss from comparisons between predicted perturbation classifications (generated by the pre-trained machine learning model) and ground truth perturbation classifications. Indeed, in one or more instances, the pre-trained machine learning model (described in relation to perturbation classification prediction fine-tuning) can include a masked autoencoder generative model trained to complete masked cellular response representations (in accordance with one or more implementations herein).

As an example, in one or more instances, the perturbation autoencoder modeling system 106 can utilize a supervised fine tuning (e.g., weakly supervised fine tuning) on the machine learning model (e.g., the generative machine learning model) to classify perturbations (e.g., gene perturbations, compound perturbations) across one or more data sets having one or more cell types. Indeed, in some cases, the perturbation autoencoder modeling system 106 can unfreeze layer norms of a generative machine learning model pre-trained for a cellular response representation completion task (in accordance with one or more embodiments herein) and fine tune (or modify) the unfrozen layer norms using a perturbation classification prediction objective (as described above).

For instance, the perturbation autoencoder modeling system 106 can fine tune the unfrozen layer norms of the (pre-trained) generative machine learning model to rebalance the generative machine learning model towards perturbation classification. As an example, the perturbation autoencoder modeling system 106 can fine-tune the generative machine learning model to balance the generative machine learning model between the cellular response representation completion task and the perturbation classification prediction task (e.g., 99% of fine-tuning for cellular response representation completion and 1% of fine-tuning for perturbation classification prediction, 90% of fine-tuning for cellular response representation completion and 10% of fine-tuning for perturbation classification prediction).

In some instances, the perturbation autoencoder modeling system 106 can further utilize average pooling of patch tokens (e.g., in lieu of the class tokens) to fine-tune one or more unfrozen layer norms of the (pre-trained) generative machine learning model on the perturbation classification prediction objective. In some instances, the perturbation autoencoder modeling system 106 can utilize the class tokens generated for the perturbation classification prediction objective to fine-tune one or more unfrozen layer norms. Furthermore, the perturbation autoencoder modeling system 106 can fine-tune (or update) the one or more unfrozen layer norms of the generative machine learning model utilizing gradient descent.

In one or more instances, the perturbation autoencoder modeling system 106 can fine-tune a variety of unfrozen layers of the (pre-trained) generative machine learning model on the perturbation classification prediction objective. For example, the perturbation autoencoder modeling system 106 can fine-tune each unfrozen layer norm in the generative machine learning model network. In some cases, the perturbation autoencoder modeling system 106 can fine-tune a subset of the unfrozen layer norm in the generative machine learning model network. Moreover, in some cases, the perturbation autoencoder modeling system 106 can fine-tune a variety of other layers (e.g., low-rank adaption layers, attention layers, multilayer perceptron), parameters (e.g., centering parameters, scaling parameters), and/or combination thereof from a (pre-trained) generative machine learning model on the perturbation classification prediction objective.

Although FIGS. 5A and 5B describe embodiments of the perturbation autoencoder modeling system 106 utilizing focused training cellular response representations (determined in accordance with one or more implementations herein), the perturbation autoencoder modeling system 106 can train a machine learning model (e.g., a generative machine learning model) on a variety of training cellular response representations via a cellular response representation completion task and subsequently training a subset of layers (or parameters) of the generative machine learning model via a perturbation classification task (as described above).

Moreover, in one or more instances, the perturbation autoencoder modeling system 106 can train a generative machine learning model in accordance with one or more implementations herein utilizing cropped cellular response representation data. For example, the perturbation autoencoder modeling system 106 can generate multiple cropped phenomic images from a phenomic image by utilizing cropped versions of the phenomic images (e.g., to train for an increasing number of epochs on a smaller training data set). The perturbation autoencoder modeling system 106 can further train the generative machine learning model utilizing the multiple cropped phenomic images in accordance with one or more implementations.

As mentioned above, the perturbation autoencoder modeling system 106 can utilize linear probing to select an inference execution layer from intermediate layers of the generative machine learning model to generate perturbation embeddings with improved accuracy via the inference execution layer. Indeed, FIGS. 6A and 6B illustrate the perturbation autoencoder modeling system 106 utilizing linear probing to select an inference execution layer from intermediate layers of the generative machine learning model to generate perturbation embeddings via the selected inference execution layer. For example, FIG. 6A illustrates the perturbation autoencoder modeling system 106 utilizing linear probing to select an inference execution layer of the generative machine learning model. In addition, FIG. 6B illustrates the perturbation autoencoder modeling system 106 utilizing the selected inference execution layer to generate one or more cellular response representation embeddings from one or more input cellular response representations.

For example, as shown in FIG. 6A, the perturbation autoencoder modeling system 106 utilizes cellular response representation(s) 604 as input for a generative machine learning model 602 to extract (or generate) cellular response representation embeddings (e.g., perturbation embeddings) from different layers (e.g., layers 1-N) of the generative machine learning model 602 in accordance with one or more implementations herein. Indeed, as shown in FIG. 6A, the perturbation autoencoder modeling system 106 generates an embedding set 1 from a layer 1, an embedding set 2 from a layer 2, and an embedding set N from an Nth layer of the generative machine learning model 602. For example, the embeddings sets 1-N can include cellular response representation embeddings generated at the particular layers of the generative machine learning model 602 for the input cellular response representation(s) 604.

In addition, as illustrated in FIG. 6A, the perturbation autoencoder modeling system 106 utilizes linear probing models 1-N with the embedding sets 1-N to generate perturbation classifications 1-N (e.g., a set of perturbation classifications for embeddings from each of the embedding sets 1-N). Indeed, the perturbation autoencoder modeling system 106 can utilize the linear probing models 1-N to generate predicted perturbation classifications that indicate particular perturbations corresponding to particular cellular response representation embeddings (e.g., the perturbation that caused the cellular response representation associated with the cellular response representation embedding). Furthermore, the perturbation autoencoder modeling system 106 determines perturbation classification accuracy metrics 1-N for the perturbation classifications 1-N that indicate an accuracy of the predicted perturbations resulting from the cellular response representation embeddings at each layer.

As further shown in an act 606 of FIG. 6A, the perturbation autoencoder modeling system 106 selects an inference execution layer from the generative machine learning model 602 by utilizing the perturbation classification accuracy metrics 1-N. In particular, in an act 608 of FIG. 6A, the perturbation autoencoder modeling system 106 compares the perturbation classification accuracy metrics 1-N to identify a layer of the generative machine learning model 602 (as the inference execution layer(s) 610) that generates cellular response representation embeddings that achieve a particular classification accuracy based on the perturbation classification accuracy metrics 1-N(e.g., a highest test balance accuracy).

In one or more instances, the perturbation autoencoder modeling system 106 utilizes the selected inference execution layer(s) 610 to generate cellular response representation embeddings (or perturbation embeddings) from input cellular response representations. For instance, as shown in FIG. 6B, the perturbation autoencoder modeling system 106 utilizes input cellular response representation(s) 612 with the selected inference execution layer 610 of the generative machine learning model 602 to generate (or extract) cellular response representation embedding(s) 614 in accordance with one or more implementations herein. Indeed, the perturbation autoencoder modeling system 106 can utilize a generative machine learning model 602 trained in accordance with one or more implementations herein.

In one or more instances, the perturbation autoencoder modeling system 106 can utilize a block-wise linear probing models that predict perturbation classifications for cellular response representation embeddings generated at various intermediate layers of the generative machine learning model. For example, the perturbation autoencoder modeling system 106 can train a linear probing model on output features (e.g., cellular response representation embeddings) of a transformer block (e.g., a layer) to predict a perturbation classification for the output features (e.g., a gene that was perturbed or the functional group that the gene belongs to). In some instances, the block-wise linear probing models can include logistic regression models.

Indeed, the perturbation autoencoder modeling system 106 can train separate linear probing models for separate layers of the generative machine learning model. For instance, the perturbation autoencoder modeling system 106 can generate a set of perturbation classification predictions using a linear probing model with a set of embeddings corresponding to an intermediate layer of the generative machine learning model. Moreover, the perturbation autoencoder modeling system 106 can modify parameters of the linear probing model by comparing the perturbation classification predictions to ground truth perturbation classifications corresponding to the cellular response representations represented by the set of embeddings.

In addition, the perturbation autoencoder modeling system 106 can generate a set of additional perturbation classification predictions using an additional linear probing model with a set of additional embeddings corresponding to an additional intermediate layer of the generative machine learning model. Furthermore, the perturbation autoencoder modeling system 106 can modify parameters of the additional linear probing model by comparing the additional perturbation classification predictions to ground truth perturbation classifications corresponding to the cellular response representations represented by the set of embeddings. Indeed, the perturbation autoencoder modeling system 106 can train various numbers of block-wise linear probing models using perturbation classification predictions generated from embeddings of various separate intermediate layers of the generative model.

In some cases, the perturbation autoencoder modeling system 106 trains a set of linear probing models to predict perturbation labels corresponding to cellular response representations utilized to generate the cellular response representation embeddings at the various intermediate layers of the generative machine learning model. In some cases, the perturbation autoencoder modeling system 106 utilizes a scaler to standardize the features of the cellular response representations (or cellular response representation embeddings) prior to training the linear probing models. For example, the perturbation autoencoder modeling system 106 utilized training data split by experiments to separate training and test data for the linear probing models.

In some instances, the perturbation autoencoder modeling system 106 utilizes a set of gene groups to train the block-wise linear probing models. For example, the perturbation autoencoder modeling system 106 can utilize a set of functionally-diverse gene groups containing a variety of genes (e.g., Anax Gene Groups). For example, the set of functionally diverse gene groups include major protein complexes (e.g. proteasome, ribosome-small/large), metabolic pathways (e.g. Krebs cycle) and signaling pathways (e.g. calcium signaling). Indeed, the training data groups can span broad biological processes that are conserved across cell types with linear separability of these groups indicating that representations are biologically meaningful regardless of cell type. In some cases, the perturbation autoencoder modeling system 106 trains the block-wise linear probing models on microscopy images of human cells of siRNA-induced gene knockdowns across four cell types (HEPG2, HUVEC, U2OS, RPE) from a dataset as described in Maciej Sypetkowski et. al., RxRx1: A Dataset for Evaluating Experimental Batch Correction Methods, Proceedings of the IEEE/CVF Conference on Computer Vision and Pattern Recognition, pp. 4284-4293 (2023) (hereinafter “RxRx1 Dataset”), which is incorporated herein by reference in its entirety.

In one or more implementations, the perturbation autoencoder modeling system 106 can utilize one or more training data sets (as described above) to fine-tune the various components of the perturbation autoencoder modeling system 106 (e.g., determining focused training cellular response representations to train the generative machine learning model via a representation completion task and/or the perturbation classification task and/or selecting an inference execution layer using linear probing models). In one or more instances, the perturbation autoencoder modeling system 106 can utilize different training data sets for fine-tuning the different components of the perturbation autoencoder modeling system 106. For example, the perturbation autoencoder modeling system 106 can determine a focused training dataset to train a generative machine learning model to unmask masked cellular response representations utilizing a first training dataset, fine-tune the generative machine learning model for a perturbation classification task utilizing a second training dataset, and training the linear probing model(s) utilizing a third training dataset. In some cases, the perturbation autoencoder modeling system 106 can utilize subsets of a training dataset to train the various components of the generative machine learning model (in accordance with one or more implementations herein).

In some instances, the perturbation autoencoder modeling system 106 can utilize different training data to train the various components of the generative machine learning model (in accordance with one or more implementations herein). As an example, the perturbation autoencoder modeling system 106 can train the generative machine learning model (via a representation completion task and/or perturbation classification task) utilizing the RxRx1 Dataset and train the linear probing models utilizing the Anax Gene Groups dataset.

Indeed, the perturbation autoencoder modeling system 106 can utilize the perturbation classification predictions from the block-wise linear probing models to determine the accuracy of cellular response representation embeddings generated at the intermediate layers of the generative machine learning model (as described above). In particular, cellular response representations of perturbed cells of the same perturbation are expected to generate similar cellular response representation embeddings. The perturbation autoencoder modeling system 106 can utilize the linear probing models to assess the accuracy of cellular response representation embeddings based on the linear probing models consistently predicting the correct (or same) perturbation from the cellular response representation embeddings (corresponding to the same perturbation) with a high accuracy.

Furthermore, the perturbation autoencoder modeling system 106 generates classification accuracy metrics for the output features of the intermediate layers using the perturbation classification predictions from the block-wise linear probing models. For example, the perturbation autoencoder modeling system 106 can determine, for each intermediate layer, a classification accuracy metric by determining the accuracy of perturbation classification predictions resulting from cellular response representation embeddings at each of the intermediate layer. In some cases, the perturbation autoencoder modeling system 106 can determine an accuracy resulting from output features (e.g., cellular response representation embeddings) by identifying a number of correct perturbation classification predictions generated from the output features (via the block-wise linear probing model). In one or more instances, the perturbation autoencoder modeling system 106 generates, for each intermediate layer, a test balanced accuracy from the perturbation classification predictions generated from the output features (via the block-wise linear probing model) as the classification accuracy metrics.

In addition, the perturbation autoencoder modeling system 106 can select an inference execution layer for the generative machine learning model utilizing the linear probing models (e.g., from the classification accuracy metrics). For example, the perturbation autoencoder modeling system 106 can rank the intermediate layers of the generative machine learning model utilizing the classification accuracy metrics determined for the intermediate layers. As an example, the perturbation autoencoder modeling system 106 can select an intermediate layer corresponding to the highest test balanced accuracy (e.g., classification accuracy metric) as the inference execution layer. For instance, the perturbation autoencoder modeling system 106 can select the inference execution layer as a block b* (for a probing task) as the block (or layer) whose output features achieve the highest text balanced accuracy when trained on the probing task, across all N blocks of the encoder (of the generative machine learning model) in accordance with the following function:

$${b}^{*} = {arg max}_{b ∈ \{ 1 , 2 , ... , N \}} BalancedAccuracy ( {z}^{( b )} ) \qquad ( 3 )$$

Indeed, in the above mentioned function (3), the perturbation autoencoder modeling system 106 can utilize z^{(b)} output features (e.g., cellular response representation embeddings) from a block b of the generative machine learning model. In some cases, the perturbation autoencoder modeling system 106 utilize the classification accuracy metric as a measure of linear separability of a feature space across experimental batches.

As an example, the perturbation autoencoder modeling system 106 can determine that an Nth intermediate layer of a generative machine learning model (e.g., block 8 out of 18, block 38 out of 48, block 20 out of 48 of the encoder) achieves a highest test balanced accuracy. Indeed, in one or more instances, the perturbation autoencoder modeling system 106 utilizes the selected inference execution layer (e.g., Nth intermediate layer) to generate cellular response representation embeddings (or perturbation embeddings) from input cellular response representations in accordance with one or more implementations herein.

Although one or more implementations herein illustrates the perturbation autoencoder modeling system 106 selecting an inference execution layer, the perturbation autoencoder modeling system 106 can select multiple inference execution layers. Indeed, the perturbation autoencoder modeling system 106 can utilize perturbation classification accuracy metrics to select multiple inference execution layers based on the inference execution layers corresponding to perturbation classification accuracy metrics that satisfy a threshold perturbation classification accuracy. For instance, the perturbation autoencoder modeling system 106 generate cellular response representation embeddings (or perturbation embeddings) from the multiple inference execution layers. For example, the perturbation autoencoder modeling system 106 can generate cellular response representation embeddings by passing forward embeddings through the multiple inference execution layers. In addition, the perturbation autoencoder modeling system 106 can generate a cellular response representation embedding from a cellular response representation by generating cellular response representation embeddings separately from the multiple inference execution layers and combining the cellular response representation embeddings (e.g., pooling, concatenation).

As mentioned above, the perturbation autoencoder modeling system 106 can utilize a trained generative machine learning model (as described herein) to generate embeddings from phenomic (microscopy) images (or other cellular response representations). In particular, FIG. 7 illustrates the perturbation autoencoder modeling system 106 utilizing a trained generative machine learning model to generate perturbation embeddings from input phenomic images. In addition, FIG. 7 also illustrates the perturbation autoencoder modeling system 106 utilizing generated perturbation embeddings to generate perturbation comparisons.

For instance, as shown in FIG. 7, the perturbation autoencoder modeling system 106, in an act 702, processes (using image processing) cell perturbations (via testing devices) to obtain phenomic images 704. In some instances, the perturbation autoencoder modeling system 106 identifies the phenomic images 704 from a repository and/or database (selected within an administrator device of the perturbation autoencoder modeling system 106 or tech-bio exploration system). Moreover, as shown in FIG. 7, the perturbation autoencoder modeling system 106 utilizes the phenomic images 704 with a generative machine learning model 706 (e.g., a masked autoencoder model (MAE) trained in accordance with one or more implementations herein). As illustrated in FIG. 7, the perturbation autoencoder modeling system 106 utilizes the generative machine learning model 706 with the phenomic images 704 to generate phenomic perturbation autoencoder embeddings 708 (i.e., cellular response representation embeddings). Moreover, as shown in FIG. 7, the perturbation autoencoder modeling system 106 can enable utilization of the phenomic perturbation autoencoder embeddings 708 with a computing device to generate various perturbation comparisons 710.

For example, the perturbation autoencoder modeling system 106 can utilize generated perturbation autoencoder embeddings to generate a variety of perturbation comparisons. To illustrate, in some cases, the perturbation autoencoder modeling system 106 can utilize generated perturbation autoencoder embeddings with perturbation databases to compare between the perturbation databases and the perturbation autoencoder embeddings to determine perturbation relationships for the perturbation autoencoder embeddings. In some cases, the perturbation autoencoder modeling system 106 can utilize the perturbation autoencoder embeddings (or generated perturbation comparisons) to generate a perturbation similarity heatmap that displays similarity measures between a plurality of perturbation autoencoder embeddings and perturbations from queried perturbation databases.

As used herein, the term “similarity measure” refers to a metric or value indicating likeness, relatedness, or similarity. For instance, a similarity measure includes a metric indicating relatedness between two perturbations (e.g., between two perturbation autoencoder embeddings). To illustrate, the perturbation autoencoder modeling system 106 can determine a similarity measure by comparing two feature vectors representing phenomic digital images. Thus, a similarity measure can include a cosine similarity between feature vectors or a measure of distance (e.g., Euclidian distance) in a feature space.

As an example, the perturbation autoencoder modeling system 106 can utilize a cosine similarity of a pair of perturbation autoencoder embeddings to generate a perturbation relationship metric. In some cases, the perturbation autoencoder modeling system 106 sets the origin of the cosine similarity space to the mean of negative experimental controls to determine perturbation comparisons between the experimental controls and the perturbation autoencoder embeddings. For instance, in some cases, the perturbation autoencoder modeling system 106 compares the determined similarities between the perturbation autoencoder embeddings with annotated relationships found in various perturbation databases. In some instances, the perturbation autoencoder modeling system 106 utilizes the perturbation autoencoder embeddings to determine cosine similarities between CRISPR knockout and/or siRNA representations in various microscopy image datasets (e.g., cell painting image datasets, cell phenotype image data sets).

For example, the perturbation autoencoder modeling system 106 can utilize various perturbation databases, such as, but not limited to, a CORUM database as described in Madalina Giurgiu et. al., CORUM: The Comprehensive Resource of Mammalian Protein Complexes, Nucleic Acids Research, 47 (Database issue): D559-D563 (2019), an hu.MAP database as described in Kevin Drew et. al., Integration of Over 9,000 Mass Spectrometry Experiments Builds a Global Map of Human Protein Complexes, Molecular Systems Biology, 13(6): 932 (2017), a Reactome database as described in Marc Gillespie et. al., The Reactome Pathway Knowledgebase 2022, Nucleic Acids Research, 50(D1): D687-D692 (2021), and a StringDB database as described in Damian Szklarczyk et. al., The STRING Database in 2021: Customizable Protein-Protein Networks, and Functional Characterization of User-Uploaded Gene/Measurement Sets, Nucleic Acids Research, 49(D1): D605-D612 (2020).

In some cases, the perturbation autoencoder modeling system 106 can apply filtering, alignment, and aggregation models to perturbation autoencoder embeddings to generate accurate perturbation-level representations for compilation into a perturbation database. Furthermore, the perturbation autoencoder modeling system 106 can identify perturbation relationships (e.g., perturbation comparisons) by accessing a database (as described above), in response to a query of one or more perturbations, and determine a similarity measure between perturbation autoencoder embeddings of the queried perturbations and the perturbations of the database. Moreover, the perturbation autoencoder modeling system 106 can utilize the similarity measures to generate perturbation similarity heatmaps. In some cases, the perturbation autoencoder modeling system 106 can apply filtering, alignment, and aggregation models to the perturbation autoencoder embeddings as described in UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY, U.S. patent application Ser. No. 18/392,989, filed Dec. 21, 2023.

For example, a perturbation similarity heatmap can include an array, table, or graphical illustration with cells representing similarity measures between perturbations. For instance, a perturbation heatmap includes a table with cells representing similarity measures at the intersection of rows representing a first set of perturbations and columns representing a second set of perturbations (based on perturbation autoencoder embeddings). In some cases, the perturbation autoencoder modeling system 106 can generate a perturbation heatmap that includes a table where rows represent individual perturbations, columns represent individual perturbations, and cells are colored to represent similarity measures for the corresponding perturbations. In some cases, the perturbation autoencoder modeling system 106 provides, for display within a graphical user interface, the perturbation similarity heatmap as the perturbation comparisons.

In some instances, the perturbation autoencoder modeling system 106 utilizes the perturbation autoencoder embeddings to determine cell counts within phenomic images (e.g., within Brightfield images, cell painted images). For example, the perturbation autoencoder modeling system 106 can utilize a classifier model to analyze the perturbation autoencoder embeddings to determine (or classify) a number of cells represented within the perturbation autoencoder embeddings. Moreover, the perturbation autoencoder modeling system 106 can utilize the predicted number of cells for the perturbation autoencoder embeddings as a cell count for the phenomic images. In some implementations, the perturbation autoencoder modeling system 106 provides, for display within a graphical user interface, the cell count within one or more phenomic images as the perturbation comparisons.

In some embodiments, the perturbation autoencoder modeling system 106 can also determine a cell type distribution from the perturbation autoencoder embeddings (for phenomic images). For example, the perturbation autoencoder modeling system 106 can utilize a classifier model to analyze the perturbation autoencoder embeddings to identify (or classify) one or more cell types present within the perturbation autoencoder embeddings. In some instances, the perturbation autoencoder modeling system 106 can further provide, for display within a graphical user interface, the distribution of cell types within one or more phenomic images as the perturbation comparisons.

In some instances, the perturbation autoencoder modeling system 106 utilizes the perturbation autoencoder embeddings with a data analysis model to generate one or more perturbation comparisons (and/or biological inferences). For instance, a data analysis model can include a computer algorithm that includes approaches, such as statistical modeling techniques, machine learning algorithms, and/or other modeling approaches to determine analyzed data (e.g., patterns, inferences, quantitative data) from input perturbation autoencoder embedding data (e.g., cellular response representation embedding data). In some cases, the perturbation autoencoder modeling system 106 utilizes a data analysis model to generate patterns, inferences, quantitative data from the perturbation autoencoder embeddings (as biological inferences). In some instances, a data analysis model includes a computer algorithm that includes approaches, such as statistical modeling techniques, machine learning algorithms, and/or other modeling approaches to perform various drug screens, compound profiling, phenoscreening, reagent profiling, and/or assay sensitivity tests from the perturbation autoencoder embeddings (of the phenomic images).

In one or more embodiments, the perturbation autoencoder modeling system 106 further utilizes batch correction transformations. For example, the perturbation autoencoder modeling system 106 applies a batch correction pipeline to generated perturbation autoencoder embeddings to eliminate unwanted batch effects and/or uncovering of the biologically relevant signals encoded in an embedding space. In some instances, the perturbation autoencoder modeling system 106 utilizes a Typical Variation Normalization (TVN) to post-process one or more perturbation autoencoder embeddings generated in accordance with one or more implementations herein. For example, the perturbation autoencoder modeling system 106 can utilize a TVN as described in D. Michael Ando et. al., Improving Phenotypic Measurements in High-Content Imaging Screens, bioRxiv, page 161422 (2017), which is incorporated herein by reference in its entirety.

In particular, the perturbation autoencoder modeling system 106 utilizes a TVN that assumes that variations observed in negative control populations are predominantly due to batch effects and estimates effective transformations based on these negative control samples. For example, the perturbation autoencoder modeling system 106 can, with TVN, fit a principal component analysis (PCA) on negative control embeddings to transform the embeddings based on the PCA kernel. Then, the perturbation autoencoder modeling system 106, for each experimental batch, utilizes a center-scale transformation on the negative control samples (on the entire batch). In some cases, the perturbation autoencoder modeling system 106 reduces the impact of axes that exhibit substantial variation (e.g., variations largely associated with unwanted batch effects) and amplifies the axes with slight variation. In addition, the perturbation autoencoder modeling system 106 utilizes a correlation alignment to further decrease batch-to-batch variation.

Indeed, by applying an aligner in the MAE-based model (trained in accordance with one or more implementations herein), the perturbation autoencoder modeling system 106 prevents (or reduces) the encoding of biologically irrelevant signals in the embedding space (for each token) via the MAE-based model. In one or more implementations, the perturbation autoencoder modeling system 106 utilizing a TVN transformation improved recall of the MAE generative model compared to utilizing no transformation approach and one or more other transformation approaches (e.g., PCA, Center by plate, Center by experiment, CenterScale by plate, PCA and CenterScale by plate, PCA and CenterScale by experiment approaches).

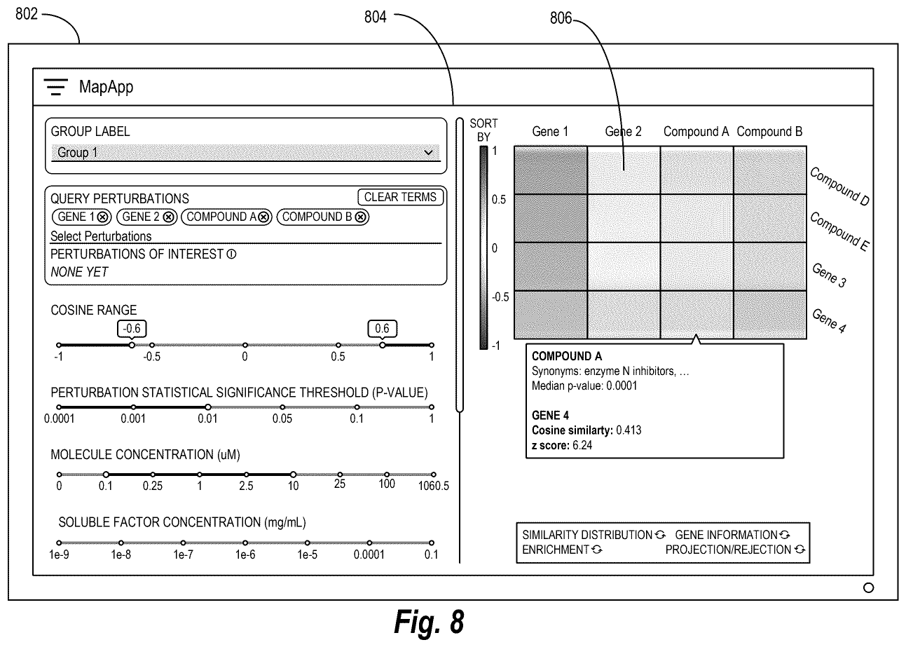

Furthermore, FIG. 8 illustrates an example of the perturbation autoencoder modeling system 106 determining perturbation comparisons from the perturbation autoencoder embeddings and providing, for display within a graphical user interface, the perturbation comparisons. Indeed, FIG. 8 illustrates an exemplary graphical user interface 804 (within a client device 802) displaying a perturbation similarity heatmap 806 (e.g., a perturbation comparison) along with user interface elements for adjusting parameters of the perturbation similarity heatmap 806 and user interface elements for displaying additional data for further analysis of the perturbation similarity heatmap 806.

As shown in FIG. 8, the perturbation autoencoder modeling system 106 can utilize the perturbation autoencoder embeddings (or perturbation comparisons from the perturbation autoencoder embeddings) to generate columns of the perturbation similarity heatmap 806. For instance, the perturbation autoencoder modeling system 106 can generate the rows of the perturbation similarity heatmap 806 with the selected perturbations identified in response to the similarity query (i.e., the returned perturbations) between one or more perturbations and the perturbation autoencoder embeddings. Indeed, as illustrated in FIG. 8, the perturbation autoencoder modeling system 106 generates the perturbation similarity heatmap 806 displaying the similarity measures between the query perturbations (from the perturbation autoencoder embeddings) (e.g., Gene1, Gene 2, Compound A, and Compound B shown as the column names of the perturbation similarity heatmap 806) and the returned perturbations (from a database of queried perturbations) (e.g., Compound D, Compound E, Gene 3, and Gene 4 shown as the row names of the perturbation similarity heatmap 806). For example, in some implementations, the perturbation autoencoder modeling system 106 compares embeddings and generates a perturbation similarity heatmap as described in UTILIZING MACHINE LEARNING MODELS TO SYNTHESIZE PERTURBATION DATA TO GENERATE PERTURBATION HEATMAP GRAPHICAL USER INTERFACES, U.S. patent application Ser. No. 18/526,707, filed Dec. 1, 2023, which is incorporated by reference in its entirety herein.

Although a particular graphical user interface is described, the perturbation autoencoder modeling system 106 can generate and/or display various graphical user interfaces to display various outcomes (or perturbation comparisons) from one or more perturbation autoencoder embeddings generated in accordance with one or more implementations herein.

In addition, in some cases, the perturbation autoencoder modeling system 106 can utilize a generative machine learning model (e.g., a MAE and/or CA-MAE model) trained in accordance with one or more embodiments herein for phenomic image correction. For instance, in some cases, the perturbation autoencoder modeling system 106 can utilize a trained generative machine learning model to generate a reconstructed (or corrected phenomic image) from an input phenomic image. As an example, the perturbation autoencoder modeling system 106 can identify a phenomic image with an imperfection or flaw (e.g., blur, smudge, dust, glare, pixel noise, or other obstruction). Then, the perturbation autoencoder modeling system 106 can utilize the phenomic image with a generative machine learning model (trained in accordance with one or more embodiments herein) to generate a corrected phenomic image that removes the imperfection or flaw in the input phenomic image.

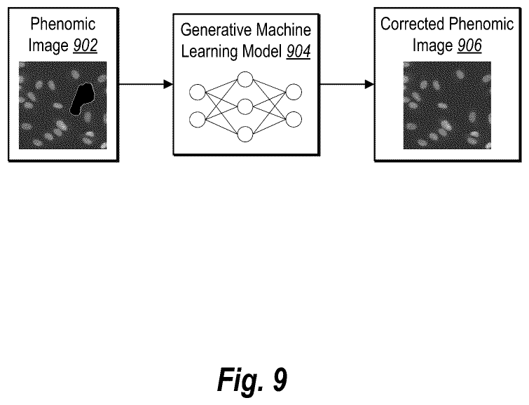

Indeed, FIG. 9 illustrates an exemplary flow of the perturbation autoencoder modeling system 106 generating a corrected phenomic image using a generative machine learning model. As shown in FIG. 9, the perturbation autoencoder modeling system 106 inputs a phenomic image 902 (e.g., an image depicting a cell phenotype with a visual obstruction) into a generative machine learning model 904 (e.g., a MAE and/or CA-MAE model trained in accordance with one or more implementations herein). As further shown in FIG. 9, the perturbation autoencoder modeling system 106 utilizes the generative machine learning model 904 to generate a corrected phenomic image 906 (e.g., via image reconstruction). As illustrated in FIG. 9, the corrected phenomic image 906 depicts the cell phenotype of the phenomic image 902 without the visual obstruction.

Furthermore, in some cases, the perturbation autoencoder modeling system 106 can utilize a generative machine learning model (e.g., a MAE and/or CA-MAE model) trained in accordance with one or more embodiments herein for phenomic image modifications (e.g., as the corrected phenomic image). For example, the perturbation autoencoder modeling system 106 can utilize the generative machine learning model to generate a corrected phenomic image that introduces cell inpainting within a phenomic image. In particular, the perturbation autoencoder modeling system 106 can utilize brightfield inputs with a generative machine learning model (trained in accordance with one or more implementations herein) to generate a cellular response representation embedding (e.g., a perturbation embedding). Moreover, the perturbation autoencoder modeling system 106 can utilize the cellular response representation embedding with a cell painting decoder to generate (or introduce) cell inpainting within a phenomic image.

In some instances, the perturbation autoencoder modeling system 106 can utilize the generative machine learning model to generate a phenomic image (e.g., via inpainting) from a phenomic image created from brightfield illumination. In particular, the perturbation autoencoder modeling system 106 can utilize the generative machine learning model (in accordance with one or more implementations) to generate accurate phenomic image embeddings from low-fidelity digital images (e.g., brightfield images) portraying different cell states over time. For example, the perturbation autoencoder modeling system 106 can identify a brightfield image that captures (at a plurality of different times) brightfield images of cells exposed to a perturbation (e.g., a CRISPR gene knockout or a pharmaceutical compound). Moreover, the perturbation autoencoder modeling system 106 can generate phenomic image embedding from the brightfield image and, subsequently, generate a predicted phenomic image from the phenomic image embedding. In some cases, the perturbation autoencoder modeling system 106 utilizes an additional machine learning model with the phenomic image embedding to decode a brightfield image into a phenomic image. In some cases, the perturbation autoencoder modeling system 106 trains a masked autoencoder generative model to mask the brightfield images of these cells, generate predicted images utilizing the masked autoencoder generative model, and compare the predicted images with the brightfield images to fine tune the masked autoencoder generative model.

Furthermore, in some cases, the perturbation autoencoder modeling system 106 can analyze new brightfield images utilizing a trained masked autoencoder generative model (in accordance with one or more implementations) and generate a phenomic image embedding (e.g., an internal feature vector from the trained autoencoder representing the cell phenotype resulting from a particular perturbation). For instance, the perturbation autoencoder modeling system 106 can utilize this phenomic image feature representation to generate various biological comparisons or predictions corresponding to the underlying perturbation portrayed in the new brightfield image.

In some instances, the perturbation autoencoder modeling system 106 can utilize different types of phenomic images, including cell paint images and/or brightfield images. The perturbation autoencoder modeling system 106 can utilize the generative machine learning model to generate a cell paint phenomic image from a brightfield phenomic image (or vice versa). For example, in some implementations, the perturbation autoencoder modeling system 106 can train a machine learning model to generate high-fidelity images (e.g., CellPaint images) from phenomic image embeddings generated by a masked autoencoder (in accordance with one or more implementations herein) from low-fidelity images (e.g., brightfield images) over time. Moreover, in some implementations the perturbation autoencoder modeling system 106 can utilize a first model to generate a color, cell paint image portraying a perturbed cell from a brightfield image portraying a perturbed cell and utilize a second model to generate a phenomic image embedding from the cell paint image portraying the perturbed cell.

Indeed, the perturbation autoencoder modeling system 106 can utilize a generative machine learning model (as described herein) to implement various tasks for brightfield images as described in GENERATING AND ANALYZING BIOLOGICAL REPRESENTATIONS UTILIZING MASKED AUTOENCODER GENERATIVE MODELS, U.S. Patent App. No. 63/690,174, filed Sep. 3, 2024, which is incorporated herein by reference in its entirety.

Although one or more embodiments of FIGS. 7-9 illustrate the perturbation autoencoder modeling system 106 utilizing phenomic perturbation autoencoder embeddings, the perturbation autoencoder modeling system 106 can generate and utilize a variety of cellular response representation embeddings (in accordance with one or more implementations herein).

Experimenters utilized an implementation of the perturbation autoencoder modeling system to assess cellular response representation embedding extraction in comparison to various existing baseline models and ablation studies. As part of the experiments, the experimenters used a variety of baseline models. For example, the experimenters utilized an implementation of visual image transformer (ViTs) encoders based on Dino-v2 backbones as described in Maxime Oquab et al., Dinov2: Learning Robust Visual Features Without Supervision (2024), a weakly supervised classifier ViT-L/16 trained using Imagenet-21k as described in Tal Ridnik et al., Imagenet-21k Pretraining For The Masses, Thirty-fifth Conference on Neural Information Processing Systems Datasets and Benchmarks Track (2021), a Masked Autoencoder ViT-L/16 trained on Imagenet-21k as described in He, and a masked autoencoder generative machine learning model MAE-ViT-L/8+ as described in Oren Kraus et al., Masked Autoencoders For Microscopy Are Scalable Learners Of Cellular Biology, Proceedings of the IEEE/CVF Conference on Computer Vision and Pattern Recognition (2024) (hereinafter “Kraus”) and U.S. patent application Ser. No. 18/545,399. In addition, the experimenters utilized various versions of the implementation of the perturbation autoencoder modeling system. For example, the experimenters utilized a channel-agnostic MAE trained in accordance with one or more implementations herein (CA-MAE-S/16), an MAE-ViT-L/8 model (as described above) trained utilizing the filtered data set (in accordance with one or more implementations herein, and an MAE-G/8 model (as described above) trained with increased model scale in terms of parameter (e.g., a 1.86 billion parameter ViT-G/8 MAE trained on Phenoprints-16M curated from filtering cellular response representations in accordance with one or more implementations herein).

For instance, FIGS. 10A and 10B illustrate experimental results of block-wise validation sets of linear probe results comparing the ViT models pretrained on cell microscopy images (left) versus natural images (right). For example, FIG. 10A illustrates results from utilizing a RxRx1 dataset and FIG. 10B illustrates results from a set of functionally-diverse gene groups containing a variety of genes (as described above). Indeed, as shown in FIGS. 10A and 10B, implementations of the perturbation autoencoder modeling system utilizing linear probing to select inference execution layers (e.g., normalized block position) over final blocks resulted in greater performance accuracy. In addition, as shown in FIGS. 10A and 10B, implementations of the perturbation autoencoder modeling system outperform other baseline models.

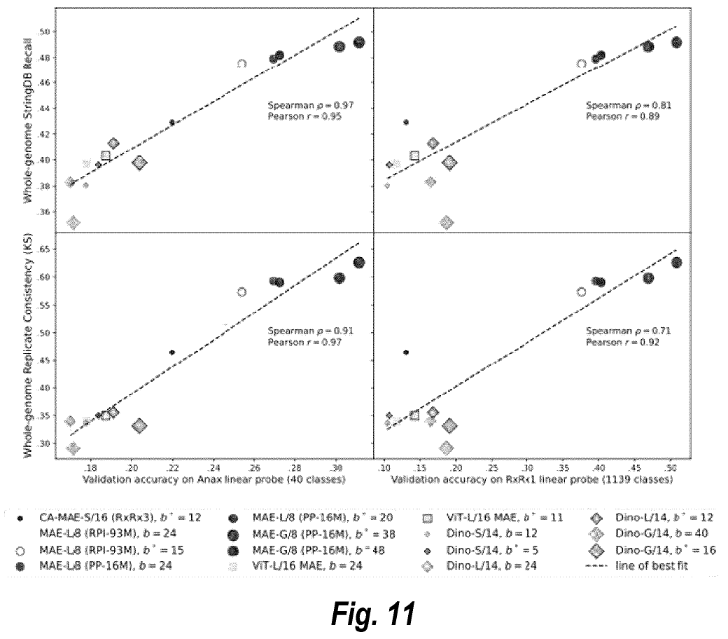

Furthermore, FIG. 11 illustrates experimental results of correlations between validation set linear probing between a selected inference execution layer (e.g., a best block) versus a last block of the various models (for both baseline models and implementations of the perturbation autoencoder modeling system 106). As shown in FIG. 11, in many cases, utilizing linear probing (as described herein) to select an inference execution layer (e.g., an intermediate layer) instead of utilizing a final block (or layer) results in improved replicate consistency (e.g., KS) and additionally improved downstream relationship recall from extracted embeddings.

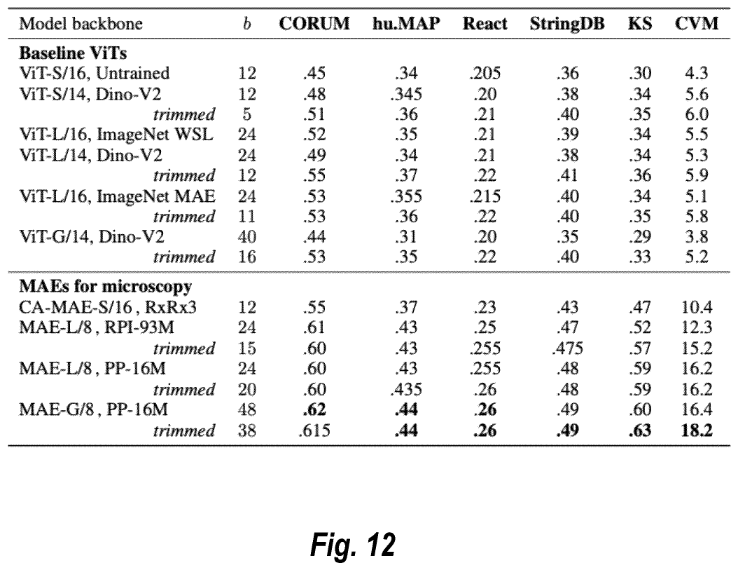

In addition, experimenters determined known biological relationship recall and univariate replicate consistency benchmarks for the various models. Indeed, the experimenters utilized linear probing to select earlier blocks as the feature encoders in the models (e.g., denoted as trimmed) with results computed over all whole-genome CRISPR knockout perturbation images in the RxRx3 dataset. Furthermore, the experimenters applied TVN and chromosome arm bias correction on the utilized data. Indeed, the biological relationship recall benchmark metrics evaluate how many annotated pair-wise relationships are recalled from public databases (CORUM, hu.MAP, Reactome-PPI (React), StringDB) based on cosine similarities of all pair-wise post-processed embeddings. Furthermore, to ensure embeddings represent technical replicates of perturbations consistently, the experimenters also evaluated model performance on replicate consistency (using test statistic that measure the difference between the perturbation replicates' similarity distribution and an empirical null distribution (with larger values indicating greater consistency). As shown in FIG. 12, an implementation of the perturbation autoencoder modeling system (e.g., MAE-G/8) outperformed the various baseline models across the public databases.

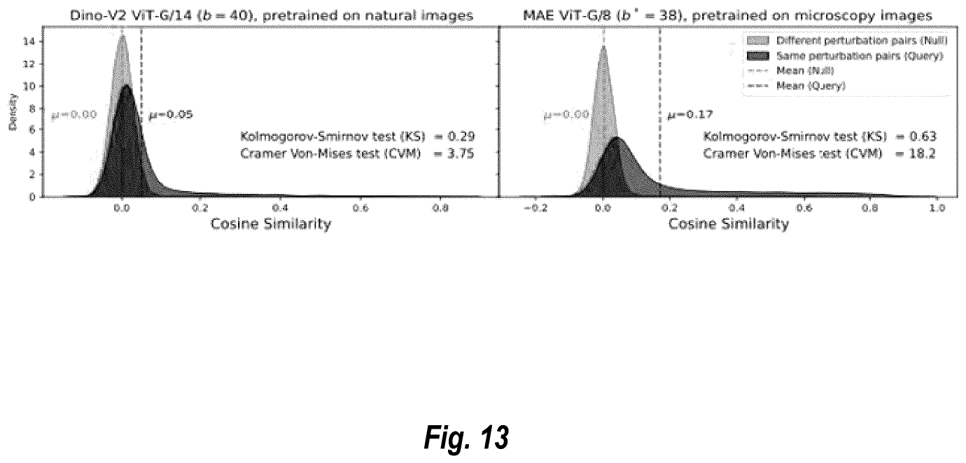

Moreover, FIG. 13 further illustrates a visualization of the replicate consistency of whole-genome results between perturbations from cosine similarity distributions on the RxRx3 dataset post-TVN between a baseline of Dino-V2 ViT-G/14 using a final block and an implementation of the perturbation autoencoder modeling system 106 (e.g., MAE-G/8 utilizing a selected inference execution layer).

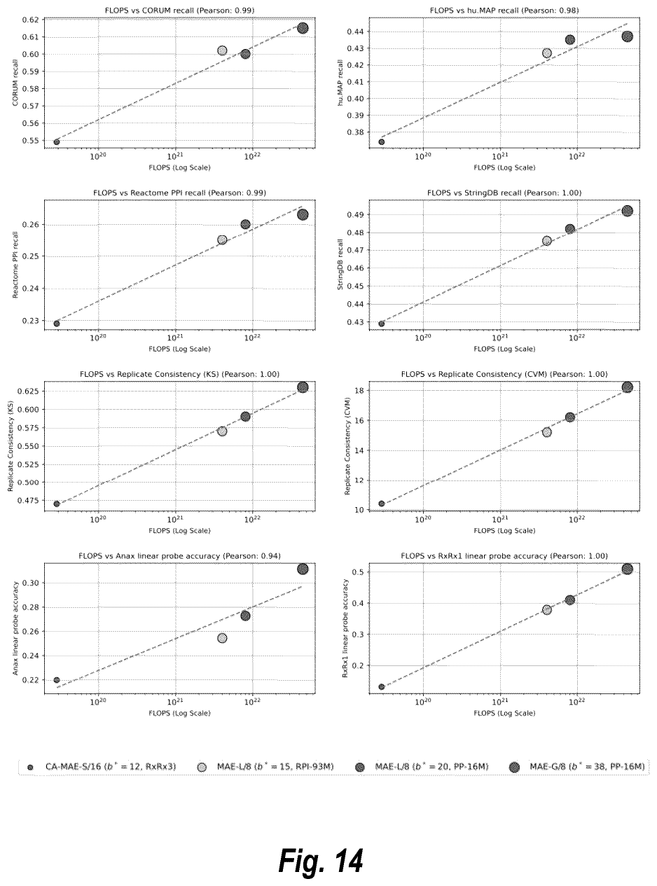

In addition, the experimenters utilized various implementations of the perturbation autoencoder modeling system with select inference blocks (or layers) b with varying training floating point operations (FLOps) and downstream recall results for various whole-genome tasks and the linear probing tasks. For instance, FIG. 14 illustrates downstream recall ratees for various whole-genome tasks and the linear probing tasks using various implementations of the perturbation encoder modeling system with selected inference blocks b (e.g., CA-MAE-S/16, MAE-L/8, and MAE-G/8) with various datasets, such as RxRx3 Dataset, RPI-93M as described in Kraus, and a curated microscopy dataset of statistically significant positive samples, PP-16M (e.g., Phenoprints-16M) curated from filtering cellular response representations in accordance with one or more implementations herein. Indeed, FIG. 14 illustrates a positive linear trend between scaling FLOps and performance improvement.

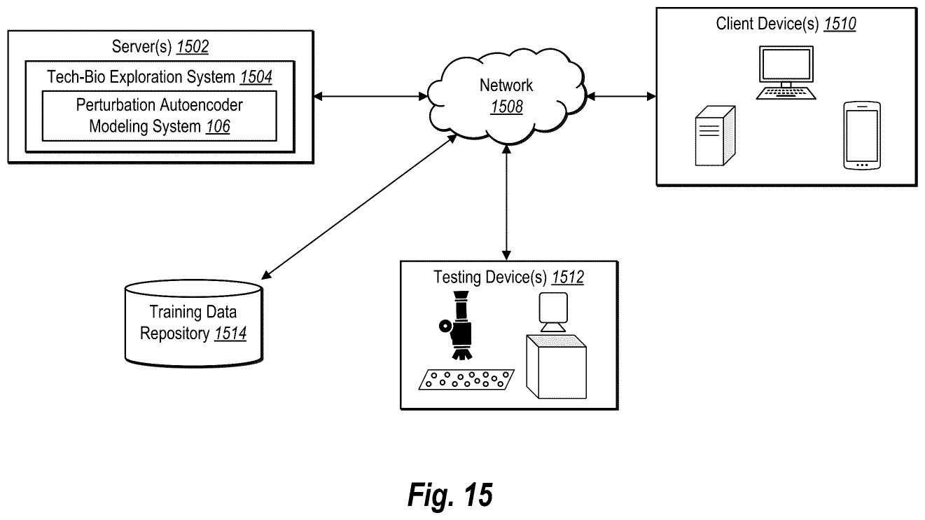

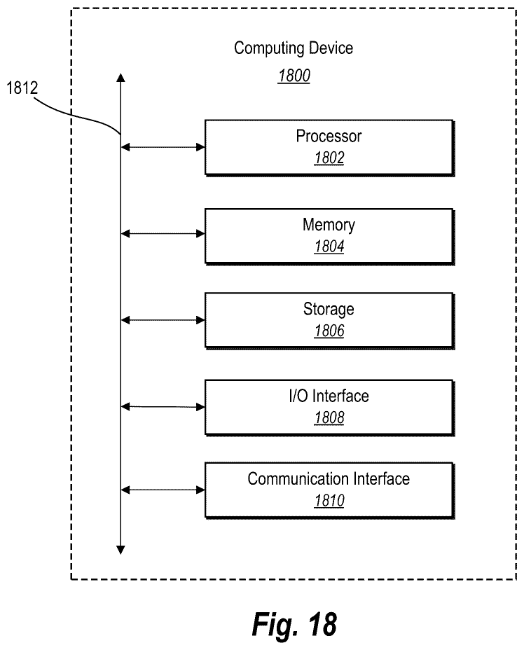

FIG. 15 illustrates a schematic diagram of a system environment in which the perturbation autoencoder modeling system 106 can operate in accordance with one or more embodiments. As shown in FIG. 15, the environment includes server(s) 1502 (which includes a tech-bio exploration system 1504 and the perturbation autoencoder modeling system 106), a network 1508, client device(s) 1510, testing device(s) 1512, and training data repository 1514. As further illustrated in FIG. 15, the various computing devices within the environment can communicate via the network 1508. Although FIG. 15 illustrates the perturbation autoencoder modeling system 106 being implemented by a particular component and/or device within the environment, the perturbation autoencoder modeling system 106 can be implemented, in whole or in part, by other computing devices and/or components in the environment (e.g., the client device(s) 1510). Additional description regarding the illustrated computing devices is provided with respect to FIG. 18 below.

As shown in FIG. 15, the server(s) 1502 can include the tech-bio exploration system 1504. In some embodiments, the tech-bio exploration system 1504 can determine, store, generate, and/or display tech-bio information including molecular compounds, phenomic images, gene knockouts, maps of biology, biology experiments from various sources, and/or machine learning tech-bio predictions. For instance, the tech-bio exploration system 1504 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal of in-vivo experiments involving chemical compounds). In one or more embodiments, the server(s) 1502 comprises a data server. In some implementations, the server(s) 1502 comprises a communication server or a web-hosting server.

For instance, the tech-bio exploration system 1504 can generate and access experimental results corresponding to gene sequences, protein shapes/folding, protein/compound interactions, phenotypes resulting from various interventions or perturbations (e.g., gene knockout sequences or compound treatments), and/or in-vivo experimentation on various treatments in living animals. By analyzing these signals (e.g., utilizing various machine learning models), the tech-bio exploration system 1504 can generate or determine a variety of predictions and inter-relationships for improving treatments/interventions.

To illustrate, the tech-bio exploration system 1504 can generate maps of biology indicating biological inter-relationships or similarities between these various input signals to discover potential new treatments. For example, the tech-bio exploration system 1504 can utilize machine learning and/or maps of biology to identify a similarity between a first gene associated with disease treatment and a second gene previously unassociated with the disease based on a similarity in resulting phenotypes from gene knockout experiments. The tech-bio exploration system 1504 can then identify new treatments based on the gene similarity (e.g., by targeting molecular compounds the impact the second gene). Similarly, the tech-bio exploration system 1504 can analyze signals from a variety of sources (e.g., protein interactions, molecular interactions, or in-vivo experiments) to predict efficacious treatments based on various levels of biological data.

The tech-bio exploration system 1504 can generate GUIs comprising dynamic user interface elements to convey tech-bio information and receive user input for intelligently exploring tech-bio information. Indeed, as mentioned above, the tech-bio exploration system 1504 can generate GUIs displaying different maps of biology that intuitively and efficiently express complex interactions between different biological systems for identifying improved treatment solutions. Furthermore, the tech-bio exploration system 1504 can also electronically communicate tech-bio information between various computing devices.

As shown in FIG. 15, the tech-bio exploration system 1504 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1504 collects, manages, and transmits data across a variety of different entities, accounts, and devices. In some cases, the tech-bio exploration system 1504 is a network system that facilitates access to (and analysis of) tech-bio information within a centralized operating system. Indeed, the tech-bio exploration system 1504 can link data from different network-based research institutions to generate and analyze maps of biology.

As also illustrated in FIG. 15, the environment includes the client device(s) 1510. For example, the client device(s) 1510 may include, but is not limited to, a mobile device (e.g., smartphone, tablet) or other type of computing device, including those explained below with reference to FIG. 18. Additionally, the client device(s) 1510 can include a computing device associated with (and/or operated by) user accounts for the tech-bio exploration system 1504. Moreover, the environment can include various numbers of client devices that communicate and/or interact with the tech-bio exploration system 1504 and/or the perturbation autoencoder modeling system 106.

Furthermore, in one or more implementations, the client device(s) 1510 includes a client application. The client application can include instructions that (upon execution) cause the client device(s) 1510 to perform various actions. For example, a user of a user account can interact with the client application on the client device(s) 1510 to access tech-bio information, initiate training of a generative machine learning model to generate cellular response representation embeddings, initiate a request for a perturbation similarity and/or generate GUIs comprising a perturbation similarity heatmap or other machine learning dataset and/or machine learning predictions/results.

As also shown in FIG. 15, the environment includes a training data repository 1514. For instance, the training data repository can include one or more computer networks, storage devices, and/or server(s) that store and/or manage training data images. As an example, the training data repository can store one or more data sets of cellular response representations (e.g., phenomic images portraying cell phenotypes, transcriptomics representations). In some cases, the training data repository 1514 can include high-content screening (HCS) microscopy data sets, such as, but not limited to, the RxRx1 dataset, RxRx3 dataset, RPI-53M dataset, and RPI-95M dataset (as described below). Indeed, HCS systems can combine automated microscopy with robotic liquid handling technologies to enable assaying cellular responses to perturbations on a massive scall (e.g., millions of cellular images across 100,000s of unique chemical and genetic perturbations).

As further shown in FIG. 15, the environment includes the network 1508. As mentioned above, the network 1508 can enable communication between components of the environment. In one or more embodiments, the network 1508 may include a suitable network and may communicate using a various number of communication platforms and technologies suitable for transmitting data and/or communication signals, examples of which are described with reference to FIG. 18. Furthermore, although FIG. 15 illustrates computing devices communicating via the network 1508, the various components of the environment can communicate and/or interact via other methods (e.g., communicate directly).

In one or more implementations, the perturbation autoencoder modeling system 106 generates and accesses machine learning objects, such as results from biological assays. As shown, in FIG. 15, the perturbation autoencoder modeling system 106 can communicate with testing device(s) 1512 to obtain and then store this information. For example, the tech-bio exploration system 1504 can interact with the testing device(s) 1512 that include intelligent robotic devices and camera devices for generating and capturing digital images of cellular phenotypes resulting from different perturbations (e.g., genetic knockouts or compound treatments of stem cells). Similarly, the testing device(s) can include camera devices and/or other sensors (e.g., heat or motion sensors) capturing real-time information from animals as part of invivo experimentation. The tech-bio exploration system 1504 can also interact with a variety of other testing device(s) such as devices for determining, generating, or extracting gene sequences or protein information.

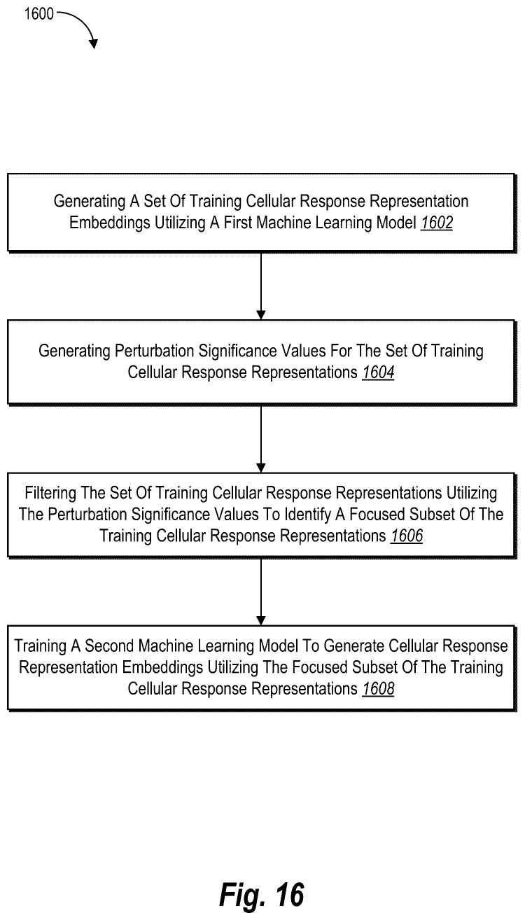

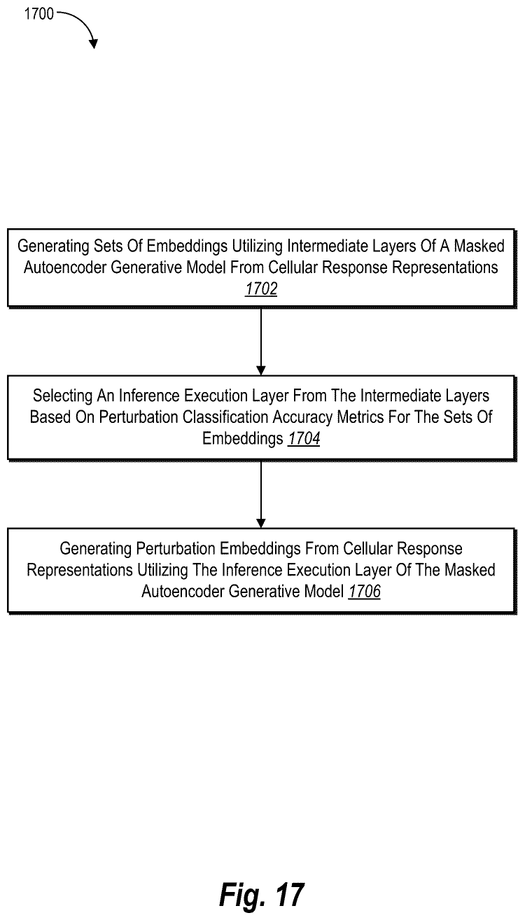

FIGS. 1-15, the corresponding text, and the examples provide a number of different systems, computer-implemented methods, and non-transitory computer readable media for generating cellular response representation embeddings in accordance with one or more implementations herein. In addition to the foregoing, embodiments can also be described in terms of flowcharts comprising acts for accomplishing a particular result. For example, FIGS. 16 and 17 illustrate flowcharts of example sequences of acts in accordance with one or more embodiments.

While FIGS. 16 and/or 17 illustrates acts according to some embodiments, alternative embodiments may omit, add to, reorder, combine, and/or modify any of the acts shown in FIGS. 16 and/or 17. The acts of FIGS. 16 and/or 17 can be performed as part of a (computer-implemented) method. Alternatively, a non-transitory computer readable medium can comprise instructions, that when executed by one or more processors, cause a computing device to perform the acts of FIGS. 16 and/or 17. In still further embodiments, a system can perform the acts of FIGS. 16 and/or 17. Additionally, the acts described herein may be repeated or performed in parallel with one another or in parallel with different instances of the same or other similar acts.

For instance, FIG. 16 illustrates an example series of acts for training a generative machine learning model to generate cellular response representation embeddings in accordance with one or more embodiments. For example, as shown in FIG. 16, the series of acts 1600 can include an act 1602 of generating a set of training cellular response representation embeddings utilizing a first machine learning model, an act 1604 of generating perturbation significance values for the set of training cellular response representations, an act 1606 of filtering the set of training cellular response representations utilizing the perturbation significance values to identify a focused subset of the training cellular response representations, and an act 1608 of training a second machine learning model to generate cellular response representation embeddings utilizing the focused subset of the training cellular response representations.

In one or more instances, the series of acts 1600 can include generating, utilizing a first machine learning model, a set of training cellular response representation embeddings from a set of training cellular response representations of cells exposed to a set of perturbations, generating perturbation significance values for the set of training cellular response representations relative to sampled subsets of the training cellular response representations, filtering the set of training cellular response representations utilizing the perturbation significance values to identify a focused subset of the training cellular response representations, and training a second machine learning model to generate cellular response representation embeddings utilizing the focused subset of the training cellular response representations.

Moreover, the series of acts 1600 can include generating a perturbation significance value by determining similarity measures between a training cellular response representation embedding corresponding to a perturbation and additional training cellular response representation embeddings from a sampled subset of the training cellular response representations corresponding to the perturbation and combining the similarity measures to generate a perturbation consistency value for the perturbation. In addition, the series of acts 1600 can include generating the perturbation significance value by comparing the perturbation consistency value for the perturbation with a null distribution of perturbation consistency values determined from a set of randomly selected training cellular response representations.

Furthermore, the series of acts 1600 can include filtering the set of training cellular response representations by comparing the perturbation significance values and a threshold perturbation significance value to identify the focused subset of the training cellular response representations.

In addition, the series of acts 1600 can include generating the set of training cellular response representation embeddings utilizing a first masked autoencoder generative model or a perturbation classification machine learning model and training the second machine learning model by training a second masked autoencoder generative model.

For example, the second machine learning model can include a masked autoencoder generative model. Moreover, the series of acts 1600 can include training the masked autoencoder generative model by generating masked training cellular response representations from the focused subset of the training cellular response representations, generating, utilizing parameters of the masked autoencoder generative model, predicted cellular response representations from the masked training cellular response representations, generating modified parameters of the masked autoencoder generative model by comparing the predicted cellular response representations to the focused subset of the training cellular response representations.

Additionally, the series of acts 1600 can include training the masked autoencoder generative model by generating, utilizing the modified parameters of the masked autoencoder generative model, a predicted perturbation class (i.e., classification) from a training cellular response representation and generating further modified parameters of the masked autoencoder generative model by comparing the predicted perturbation class with a ground truth perturbation class for the training cellular response representation. Moreover, the series of acts 1600 can include generating the further modified parameters by freezing a first set of layers of the masked autoencoder generative model comprising a subset of the modified parameters trained from the masked training cellular response representations and modifying a second set of layers by comparing the predicted perturbation class with the ground truth perturbation class to generate the further modified parameters.

Furthermore, FIG. 17 illustrates an example series of acts for selecting an inference execution layer to generate cellular response representation embeddings from a masked autoencoder generative model in accordance with one or more embodiments. For instance, as shown in FIG. 17, the series of acts 1700 include an act 1702 of generating sets of embeddings utilizing intermediate layers of a masked autoencoder generative model from cellular response representations, an act 1704 of selecting an inference execution layer from the intermediate layers based on perturbation classification accuracy metrics for the sets of embeddings, and an act 1706 of generating perturbation embeddings from cellular response representations utilizing the inference execution layer of the masked autoencoder generative model.

For example, the series of acts 1700 can include generating, utilizing a first intermediate layer of a masked autoencoder generative model, a first set of embeddings from a set of cellular response representations of perturbed cells, generating, utilizing a second intermediate layer of the masked autoencoder generative model, a second set of embeddings from the set of cellular response representations of the perturbed cells, selecting, utilizing linear probing models, an inference execution layer from the first intermediate layer and the second intermediate layer based on perturbation classification accuracy metrics for the first set of embeddings and the second set of embeddings, and generating, utilizing the inference execution layer of the masked autoencoder generative model, cellular response representation embeddings from cellular response representations of a plurality of perturbed cells.

Moreover, the series of acts 1700 can include training a first linear probing model, from the linear probing models, by generating, utilizing the first linear probing model, a first set of perturbation classification predictions from the first set of embeddings corresponding to the first intermediate layer and modifying parameters of the first linear probing model based on a comparison between the first set of perturbation classification predictions and a first set of ground truth perturbation classifications. Furthermore, the series of acts 1700 can include training a second linear probing model, from the linear probing models, by generating, utilizing the second linear probing model, a second set of perturbation classification predictions from the second set of embeddings corresponding to the second intermediate layer and modifying parameters of the second linear probing model based on a comparison between the second set of perturbation classification predictions and a second set of ground truth perturbation classifications.

In addition, the series of acts 1700 can include determining the perturbation classification accuracy metrics by generating, for the first intermediate layer, a first perturbation classification accuracy metric from the first set of embeddings utilizing a first linear probing model and generating, for the second intermediate layer, a second perturbation classification accuracy metric from the second set of embeddings utilizing a second linear probing model. Moreover, the series of acts 1700 can include generating the first perturbation classification accuracy metric by generating a first set of perturbation classification predictions from the first set of embeddings utilizing the first linear probing model and determining the first perturbation classification accuracy metric from the first set of perturbation classification predictions. In addition, the series of acts 1700 can include selecting the inference execution layer by selecting between the first intermediate layer and the second intermediate layer based on a comparison of the first perturbation classification accuracy metric and the second perturbation classification accuracy metric.

Additionally, the series of acts 1700 can include selecting the inference execution layer utilizing the linear probing models by selecting the inference execution layer utilizing logistic regression models that predict perturbation classifications from cellular response representation embeddings.

Moreover, in some cases, the cellular response representations can include phenomic images portraying the perturbed cells. In addition, the series of acts 1700 can include generating, utilizing the inference execution layer of the masked autoencoder generative model, the cellular response representation embeddings from the phenomic images portraying the perturbed cells.

Additionally, the series of acts 1700 can include generating perturbation similarity metrics utilizing the cellular response representation embeddings by identifying a first cellular response representation embedding of the cellular response representation embeddings corresponding to a first perturbation applied to a first cell, identifying a second cellular response representation embedding of the cellular response representation embeddings corresponding to a second perturbation applied to a second cell, and comparing the first cellular response representation embedding and the second cellular response representation embedding to determine a perturbation similarity metric between the first perturbation and the second perturbation.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 18 illustrates a block diagram of an example computing device 1800 that may be configured to perform one or more of the processes described above. One will appreciate that one or more computing devices, such as the computing device 1800 may represent the computing devices described above. In one or more embodiments, the computing device 1800 may be a mobile device (e.g., a mobile telephone, a smartphone, a PDA, a tablet, a laptop, a camera, a tracker, a watch, a wearable device, etc.). In some embodiments, the computing device 1800 may be a non-mobile device (e.g., a desktop computer or another type of client device). Further, the computing device 1800 may be a server device that includes cloud-based processing and storage capabilities.

As shown in FIG. 18, the computing device 1800 can include one or more processor(s) 1802, memory 1804, a storage device 1806, input/output interfaces 1808 (or “I/O interfaces 1808”), and a communication interface 1810, which may be communicatively coupled by way of a communication infrastructure (e.g., bus 1812). While the computing device 1800 is shown in FIG. 18, the components illustrated in FIG. 18 are not intended to be limiting. Additional or alternative components may be used in other embodiments. Furthermore, in certain embodiments, the computing device 1800 includes fewer components than those shown in FIG. 18. Components of the computing device 1800 shown in FIG. 18 will now be described in additional detail.

In particular embodiments, the processor(s) 1802 includes hardware for executing instructions, such as those making up a computer program. As an example, and not by way of limitation, to execute instructions, the processor(s) 1802 may retrieve (or fetch) the instructions from an internal register, an internal cache, memory 1804, or a storage device 1806 and decode and execute them.

The computing device 1800 includes memory 1804, which is coupled to the processor(s) 1802. The memory 1804 may be used for storing data, metadata, and programs for execution by the processor(s). The memory 1804 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1804 may be internal or distributed memory.

The computing device 1800 includes a storage device 1806 includes storage for storing data or instructions. As an example, and not by way of limitation, the storage device 1806 can include a non-transitory storage medium described above. The storage device 1806 may include a hard disk drive (HDD), flash memory, a Universal Serial Bus (USB) drive or a combination these or other storage devices.

As shown, the computing device 1800 includes one or more I/O interfaces 1808, which are provided to allow a user to provide input to (such as user strokes), receive output from, and otherwise transfer data to and from the computing device 1800. These I/O interfaces 1808 may include a mouse, keypad or a keyboard, a touch screen, camera, optical scanner, network interface, modem, other known I/O devices or a combination of such I/O interfaces 1808. The touch screen may be activated with a stylus or a finger.

The I/O interfaces 1808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1808 are configured to provide graphical data to a display for presentation to a user. The graphical data may be representative of one or more graphical user interfaces and/or any other graphical content as may serve a particular implementation.

The computing device 1800 can further include a communication interface 1810. The communication interface 1810 can include hardware, software, or both. The communication interface 1810 provides one or more interfaces for communication (such as, for example, packet-based communication) between the computing device and one or more other computing devices or one or more networks. As an example, and not by way of limitation, communication interface 1810 may include a network interface controller (NIC) or network adapter for communicating with an Ethernet or other wire-based network or a wireless NIC (WNIC) or wireless adapter for communicating with a wireless network, such as a WI-FI. The computing device 1800 can further include a bus 1812. The bus 1812 can include hardware, software, or both that connects components of computing device 1800 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1800 can include a client device that includes a requester application or a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system (e.g., the tech-bio exploration system 1504) may include a variety of servers, sub-systems, programs, modules, logs, and data stores. In particular embodiments, the tech-bio exploration system may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system may also include suitable components such as network interfaces, security mechanisms, load balancers, failover servers, management-and-network-operations consoles, other suitable components, or any suitable combination thereof. In particular embodiments, the tech-bio exploration system may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system and one or more client devices. An action logger may be used to receive communications from a web server about a user's actions on or off the tech-bio exploration system. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system. A privacy setting of a user determines how particular information associated with a user can be shared. The authorization server may allow users to opt in to or opt out of having their actions logged by the tech-bio exploration system or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

1. A computer-implemented method comprising:
generating a set of training cellular response representations from microscopy images of cells exposed to a set of perturbations;
generating, utilizing a first machine learning model, a set of training cellular response representation embeddings from the set of training cellular response representations by embedding the set of training cellular response representations in a machine learning feature space as vector representations;
generating perturbation significance values for the set of training cellular response representations relative to sampled subsets of the training cellular response representations by comparing, within the machine learning feature space, training cellular response representation embeddings from the set of training cellular response representation embeddings;
filtering the set of training cellular response representations utilizing the perturbation significance values corresponding to the set of training cellular response representation embeddings to identify a focused subset of the training cellular response representations for a staged training process;
generating masked training cellular response representations from the focused subset of the training cellular response representations; and
training a second machine learning model via the staged training process to generate cellular response representation embeddings for generating biological inferences in a drug discovery pipeline by:
modifying parameters of the second machine learning model during a cellular response representation completion training stage utilizing the masked training cellular response representations corresponding to the focused subset of the training cellular response representations;
identifying a first set of layers of the second machine learning model and a second set of layers of the second machine learning model;
freezing the first set of layers of the second machine learning model during a perturbation classification training stage to preserve learned parameters from the cellular response representation completion training stage; and
selectively modifying the second set of layers of the second machine learning model during the perturbation classification training stage utilizing a perturbation classification task.

2. The computer-implemented method of claim 1, further comprising generating a perturbation significance value by:
determining similarity measures between a training cellular response representation embedding corresponding to a perturbation and additional training cellular response representation embeddings from a sampled subset of the training cellular response representations corresponding to the perturbation; and
combining the similarity measures to generate a perturbation consistency value for the perturbation.

3. The computer-implemented method of claim 2, further comprising generating the perturbation significance value by comparing the perturbation consistency value for the perturbation with a null distribution of perturbation consistency values determined from a set of randomly selected training cellular response representations.

4. The computer-implemented method of claim 1, further comprising filtering the set of training cellular response representations by comparing the perturbation significance values and a threshold perturbation significance value to identify the focused subset of the training cellular response representations.

5. The computer-implemented method of claim 1, further comprising:
generating the set of training cellular response representation embeddings utilizing a first masked autoencoder generative model or a perturbation classification machine learning model; and
training the second machine learning model by training a second masked autoencoder generative model.

6. The computer-implemented method of claim 1, wherein the second machine learning model comprises a masked autoencoder generative model and further comprising training the masked autoencoder generative model by:
generating the masked training cellular response representations from the focused subset of the training cellular response representations;
generating, utilizing parameters of the masked autoencoder generative model, predicted cellular response representations from the masked training cellular response representations; and
modifying the parameters of the masked autoencoder generative model by comparing the predicted cellular response representations to the focused subset of the training cellular response representations.

7. The computer-implemented method of claim 6, further comprising training the masked autoencoder generative model by:
generating, utilizing the modified parameters of the masked autoencoder generative model, a predicted perturbation class from the training cellular response representation; and
modifying the second set of layers of the masked autoencoder generative model by comparing the predicted perturbation class with a ground truth perturbation class for the training cellular response representation.

8. The computer-implemented method of claim 7, further comprising training the masked autoencoder generative model by:
freezing the first set of layers of the masked autoencoder generative model trained from the masked training cellular response representations; and
modifying a subset of the parameters by modifying the second set of layers of the masked autoencoder generative model by comparing the predicted perturbation class with the ground truth perturbation class.

9. A system comprising:
at least one processor; and
at least one non-transitory computer-readable storage medium storing instructions that, when executed by the at least one processor, cause the system to:
generate a set of training cellular response representations from microscopy images of cells exposed to a set of perturbations;
generate, utilizing a first machine learning model, a set of training cellular response representation embeddings from the set of training cellular response representations by embedding the set of training cellular response representations in a machine learning feature space as vector representations;
generate perturbation significance values for the set of training cellular response representations relative to sampled subsets of the training cellular response representations by comparing, within the machine learning feature space, training cellular response representation embeddings from the set of training cellular response representation embeddings;
filter the set of training cellular response representations utilizing the perturbation significance values corresponding to the set of training cellular response representation embeddings to identify a focused subset of the training cellular response representations for a staged training process;
generate masked training cellular response representations from the focused subset of the training cellular response representations; and
train a second machine learning model via the staged training process to generate cellular response representation embeddings for generating biological inferences in a drug discovery pipeline by:
modifying parameters of the second machine learning model during a cellular response representation completion training stage utilizing the masked training cellular response representations corresponding to the focused subset of the training cellular response representations;
identifying a first set of layers of the second machine learning model and a second set of layers of the second machine learning model;
freezing the first set of layers of the second machine learning model during for a perturbation classification training stage to preserve learned parameters from the cellular response representation completion training stage; and
selectively modifying the second set of layers of the second machine learning model during the perturbation classification training stage utilizing a perturbation classification task.

10. The system of claim 9, wherein the instructions cause the system to generate a perturbation significance value by:
determining similarity measures between a training cellular response representation embedding corresponding to a perturbation and additional training cellular response representation embeddings from a sampled subset of the training cellular response representations corresponding to the perturbation; and
combining the similarity measures to generate a perturbation consistency value for the perturbation.

11. The system of claim 10, wherein the instructions cause the system to generate the perturbation significance value by comparing the perturbation consistency value for the perturbation with a null distribution of perturbation consistency values determined from a set of randomly selected training cellular response representations.

12. The system of claim 9, wherein the instructions cause the system to:
generate, utilizing the second machine learning model with a phenomic image portraying a cell, a cellular response representation embedding for the phenomic image;
determine one or more similarity measures between the cellular response representation embedding and one or more additional cellular response representation embeddings; and
provide, for display within a graphical user interface of a client device, the one or more similarity measures.

13. The system of claim 9, wherein the second machine learning model comprises a masked autoencoder generative model and wherein the instructions cause the system to train the masked autoencoder generative model by:
generating the masked training cellular response representations from the focused subset of the training cellular response representations;
generating, utilizing the parameters of the masked autoencoder generative model, predicted cellular response representations from the masked training cellular response representations; and
modifying the parameters of the masked autoencoder generative model by comparing the predicted cellular response representations to the focused subset of the training cellular response representations.

14. The system of claim 13, wherein the instructions cause the system to train the masked autoencoder generative model by:
generating, utilizing modified parameters of the masked autoencoder generative model, a predicted perturbation class from a training cellular response representation; and
modifying a subset of the parameters of the masked autoencoder generative model by comparing the predicted perturbation class with a ground truth perturbation class for the training cellular response representation.

15. A non-transitory computer-readable medium storing instructions that, when executed by at least one processor, cause a computing device to:
generate a set of training cellular response representations from transcriptomic profiles of cells exposed to a set of perturbations;
generate, utilizing a first machine learning model, a set of training cellular response representation embeddings from the set of training cellular response representations by embedding the set of training cellular response representations in a machine learning feature space as vector representations;
generate perturbation significance values for the set of training cellular response representations relative to sampled subsets of the training cellular response representations by comparing, within the machine learning feature space, training cellular response representation embeddings from the set of training cellular response representation embeddings;
filter the set of training cellular response representations utilizing the perturbation significance values corresponding to the set of training cellular response representation embeddings to identify a focused subset of the training cellular response representations for a staged training process;
generate masked training cellular response representations from the focused subset of the training cellular response representations; and
train a second machine learning model via the staged training process to generate cellular response representation embeddings for generating biological inferences in a drug discovery pipeline by:
modifying parameters of the second machine learning model during a cellular response representation completion training stage utilizing the masked training cellular response representations corresponding to the focused subset of the training cellular response representations;
identifying a first set of layers of the second machine learning model and a second set of layers of the second machine learning model;
freezing the first set of layers of the second machine learning model during a perturbation classification training stage to preserve learned parameters from the cellular response representation completion training stage; and
selectively modifying the second set of layers of the second machine learning model during the perturbation classification training stage utilizing a perturbation classification task.

16. The non-transitory computer-readable medium of claim 15, wherein the instructions cause the computing device to generate a perturbation significance value by:
determining similarity measures between a training cellular response representation embedding corresponding to a perturbation and additional training cellular response representation embeddings from a sampled subset of the training cellular response representations corresponding to the perturbation; and
combining the similarity measures to generate a perturbation consistency value for the perturbation.

17. The non-transitory computer-readable medium of claim 16, wherein the instructions cause the computing device to generate the perturbation significance value by comparing the perturbation consistency value for the perturbation with a null distribution of perturbation consistency values determined from a set of randomly selected training cellular response representations.

18. The non-transitory computer-readable medium of claim 15, wherein the instructions cause the computing device to filter the set of training cellular response representations by comparing the perturbation significance values and a threshold perturbation significance value to identify the focused subset of the training cellular response representations.

19. The non-transitory computer-readable medium of claim 15, wherein the second machine learning model comprises a masked autoencoder generative model and wherein the instructions cause the computing device to train the masked autoencoder generative model by:
generating the masked training cellular response representations from the focused subset of the training cellular response representations;
generating, utilizing the parameters of the masked autoencoder generative model, predicted cellular response representations from the masked training cellular response representations; and
modifying the parameters of the masked autoencoder generative model by comparing the predicted cellular response representations to the focused subset of the training cellular response representations.

20. The non-transitory computer-readable medium of claim 19, wherein the instructions cause the computing device to train the masked autoencoder generative model by:
generating, utilizing the modified parameters of the masked autoencoder generative model, a predicted perturbation class from a training cellular response representation; and
modifying the second set of layers of the masked autoencoder generative model by comparing the predicted perturbation class with a ground truth perturbation class for the training cellular response representation.
